(19) United States
(12) Patent Application Publication
(10) Pub. No.: US 2024/0342085 A1
(43) Pub. Date: Oct. 17, 2024

(54) STIMULI-INDUCED DELIVERY SYSTEMS

(71) Applicant: RAMOT AT TEL-AVIV UNIVERSITY LTD., Tel Aviv (IL)

(72) Inventors: Roey Jacob AMIR, Tel Aviv (IL); Gadi SLOR, Tel Aviv (IL); Shahar TEVET, Tel Aviv (IL)

(21) Appl. No.: 18/580,272

(22) PCT Filed: Jul. 17, 2022
(86) PCT No.: PCT/IL2022/050770

§ 371 Date: Jan. 18, 2024

Related U.S. Application Data
(60) Provisional application No. 63/223,014, filed on Jul. 18, 2021.

(51) Int. Cl. A61K 9/107; A61K 8/02; A61K 8/81; A61K 47/32

(52) U.S. Cl. CPC A61K 9/1075; A61K 8/0291; A61K 8/8164; A61K 47/32

(57) ABSTRACT

The present invention provides a stimuli-induced delivery system comprising self¬assembled amphiphilic tri-block copolymers in the form of micelles. The delivery system enables the release of a cargo from within the micelles following a transition to di-block copolymers micelles and subsequent activation by a protein to induce disassembly of the micelles.

### FIELD OF INVENTION

The present invention relates to stimuli-induced delivery systems comprising self-assembled amphiphilic tri-block copolymers and methods of use thereof for controlled release of an active agent at a target site.

### BACKGROUND

Stimuli-responsive polymeric micelles have great potential to serve as smart drug delivery systems (DDSs). To reach their target tissue without premature release of their therapeutic cargo, micelles must be highly stable towards dilution and non-specific interactions with serum proteins and endothelial cells. At the same time, micelles must also disassemble once reaching their target site in order to release their therapeutic cargo. Furthermore, micellar degradation and disassembly are critical for traceless clearance of the DDSs from the body after delivering their payload. The high specificity and over-expression of disease-associated enzymes in diseased tissues, make enzymes highly promising stimuli for triggering the selective release of drugs from micellar nanocarriers. Unlike stimuli-responsive micelles that respond to dimensionless stimuli such as light or temperature, enzymatically degradable micelles, show reverse correlation between micellar stability and their responsiveness to the activating enzymes. Once micellar stability reaches a certain threshold, they become unreactive towards the activating enzyme. The challenge to balance between stability and degradability is one of the key limitations of such nanocarriers.

Nanocarriers composed of self-assembled amphiphiles have been demonstrated. The amphiphiles self-assemble into thermodynamically stable micelles, thereby enabling the control of the disassembly and release profiles of a cargo which may be conjugated to the amphiphiles or encapsulated within the self-assembled micelles.

WO 2016/038595 describes an enzymatic stimuli-responsive amphiphilic hybrid delivery system in micellar form, based on a hydrophilic polyethylene glycol (PEG) polymer conjugated to a hydrophobic dendron. The delivery system disassembles upon enzymatic stimuli/cleavage.

WO 2016/038596 describes an enzyme- or pH-responsive amphiphilic hybrid delivery system in micellar form for delivery of agrochemicals, based on a hydrophilic polyethylene glycol (PEG) polymer conjugated to a hydrophobic dendron. The delivery system disassembles upon enzymatic trigger or pH-based stimuli.

U.S. 2017/0035916 describes an enzymatic stimuli-responsive amphiphilic delivery system in micellar form, comprising at least one hybrid polymer or a mixture of polymers, each polymer is based on a hydrophilic polyethylene glycol (PEG) polymer conjugated to a hydrophobic dendron and a labeling moiety selected from a fluorescent dye, a dark quencher and a fluorinated moiety that acts as a magnetic probe for turn on/off of ¹⁹F-magnetic resonance (MR) signal.

While the majority of the amphiphiles are based on diblock copolymers, several studies have designed triblock copolymers structures. Cambón et al., (RSC Adv. 2014, 4 (105), 60484-60496) studied the self-assembly process of two reverse triblock poly(butylene oxide)-poly(ethylene oxide)-poly(butylene oxide) block copolymers, BO₈EO₉₀BO₈ and BO₂₀EO₄₁₁BO₂₀, useful as drug delivery nanocarriers. Lundberg et al., (Soft Matter 2013, 9 (1), 82-89) studied the effect of pH on the self-assembly of histamine functionalized poly(allyl glycidyl ether)-b-poly(ethylene glycol)-b-poly(allyl glycidyl ether) (PAGE-PEO-PAGE) triblock copolymers. Peters et al., (Macromolecules 2017, 50 (16), 6303-6313) studied the equilibrium chain exchange of asymmetric B₁AB₂ and AB₁B₂ branched triblock copolymers in a B selective solvent via dissipative particle dynamics simulations. Lu at al., (Macromolecules 2015, 48 (8), 2667-2676) studied the effect of polymer architecture on molecular exchange in block copolymer micelles (notably PEP-PS-PEP and PS-PEP-PS triblock copolymers, where PS and PEP refer to poly(styrene) and poly(ethylene-alt-propylene), respectively) using time-resolved small-angle neutron scattering (TR-SANS). Wu et al., (Colloids Interface Sci. Commun. 2021, 41, 100386) studied the stability of flower micelles vs. traditional micelles in brine and serum. The micelles were formed by the self-assembly of triblock and diblock copolymers of polyethylene glycol (PEG) and poly(ε-caprolactone) (PCL) in water.

Decreasing the overall molecular weight of polymeric amphiphiles while preserving their hydrophilic to lipophilic balance (HLB), was found to significantly reduce micellar stability towards enzymatic degradation (Slor et al., Chem. Commun. 2018, 54 (50), 6875-6878; Gao et al., Chem. Sci. 2019, 10 (10), 3018-3024; and Rosenbaum et al., Biomacromolecules 2017, 18 (10), 3457-3468). There is a great unmet need for amphiphile assemblies that overcome the stability-responsiveness barrier.

### SUMMARY

The present invention provides an amphiphilic polymeric delivery system in micellar form, based on a hydrophobic-hydrophilic-hydrophobic (B-A-B) tri-block copolymer (TBC) comprising a stimuli-responsive linker located at the center of the hydrophilic block, wherein upon activation by external stimuli, the linker undergoes cleavage to afford two hydrophobic-hydrophilic (B-A′) di-block copolymer (DBC) amphiphiles having substantially the same hydrophilic to lipophilic balance (HLB) as the tri-block copolymer, the di-block copolymers can undergo a subsequent enzymatic cleavage of the hydrophobic end groups to result in disassembly of the micellar structure and release of the cargo encapsulated therein or attached thereto.

Disclosed herein for the first time is the introduction of a stimuli-responsive linker at the center of an enzyme/protein-responsive hydrophobic-hydrophilic-hydrophobic (B-A-B) tri-block copolymer (TBC) amphiphile. According to the principles of the present invention, activation of the linker by a stimulus results in an architectural transition from the TBC to two hydrophilic-hydrophobic (A′-B) di-block copolymer (DBC) amphiphiles. This architectural transition has surprisingly been found to markedly increase the responsiveness of the amphiphiles to degrading enzymes whereby the di-block copolymer amphiphiles are substantially more prone to enzymatic degradation.

In particular, the tri-block amphiphiles of the present invention are designed to undergo on-demand splitting in the middle of the hydrophilic (A) block, yielding two amphiphilic di-block copolymers having substantially the same hydrophilic to lipophilic balance (HLB) as the parent tri-block amphiphiles. In this manner, the di-block copolymers remain assembled as micelles with minor to negligible release of the cargo encapsulated therein or attached thereto. However, by lowering the molecular weight of the tri-block amphiphiles due to the splitting and architectural change from tri-block to di-block copolymers, a significant increase in the unimer-micelle exchange rate occurs. This change in unimer-micelle equilibrium facilitates the enzymatic degradation of the di-block copolymers, leading to complete disassembly of the hydrolyzed polymers (FIG. 1). The ability to control the responsiveness of amphiphiles towards enzymatic degradation by introducing a stimuli-responsive cleavage site within the hydrophilic block, enables the modular design of highly stable and yet enzyme-responsive polymeric nano-assemblies that overcome the stability-responsiveness barrier.

According to a first aspect, there is provided a delivery system in micellar form comprising a self-assembled amphiphilic tri-block copolymer comprising two substantially identical hydrophobic segments each comprising at least one protein cleavable site and a hydrophilic segment therebetween, wherein the hydrophilic segment comprises a central stimulus-responsive linker which undergoes cleavage upon a stimulus thereby forming two di-block copolymers having substantially the same hydrophilic to hydrophobic ratio as the amphiphilic tri-block copolymer.

In one embodiment, the delivery system further comprises a cargo encapsulated within the micelles. In another embodiment, the delivery system further comprises a cargo covalently linked to the hydrophobic segments. In further embodiments, the cargo is selected from a pharmaceutical active ingredient, an agrochemical agent, a cosmetic agent, an imaging agent, and a diagnostic agent. Each possibility represents a separate embodiment.

In some embodiments, the hydrophilic segment comprises polyacrylic acid, poly(hydroxyethyl acrylate), polyethylene glycol (PEG), poly(oligo-ethylene glycol acrylate), polyacrylamide, polymethyl oxazoline, polyethyl oxazoline, polysarcosine, polypeptide, polypeptoid, hydrophilic polymethacrylate, polyamine, hydrophilic nylon, polyvinyl alcohol, hydrophilic protein, or polycarbohydrate. Each possibility represents a separate embodiment. In other embodiments, the hydrophilic segment comprises polyacrylic acid, poly(2-hydroxyethyl acrylate), polyethylene glycol (PEG), or poly(oligo-ethylene glycol acrylate). Each possibility represents a separate embodiment. In particular embodiments, the hydrophilic segment has a molecular weight of about 0.5 to about 100 kDa, including each value within the specified range. In further embodiments, the hydrophilic segment is linked to the hydrophobic segments by a group selected from the group consisting of —Z—, —X¹—Z—X²—, —Z¹—X¹—Z²—X²—, wherein Z, Z¹, and Z² are each independently selected from C₁-C₁₀ alkylene, C₂-C₁₀ alkenylene, C₂-C₁₀ alkynylene, and arylene; X, X¹, and X² are each independently selected from O, S and NH; —O—; —S—; —NH—; —C(═O)—; —C(═O)—O—; —O—C(═O)—O—; —C(═O)—NH—; —NH—C(═O)—NH—; —NH—C(═O)—O—; —S(═O)—; —S(═O)—O—; —PO(═O)—O—; triazolylene, and any combination thereof. Each possibility represents a separate embodiment.

In various embodiments, the linker in the hydrophilic segment is responsive to a stimulus. In certain embodiments, the stimulus is chemically-induced. In other embodiments, the stimulus is physically-induced. In particular embodiments, the stimulus comprises a change in at least one of temperature, pH, light (UV light or near infrared light), and electric field. Each possibility represents a separate embodiment. In other embodiments, the stimulus comprises the addition of a redox agent or an activating enzyme. Each possibility represents a separate embodiment.

In further embodiments, the stimulus-responsive linker comprises a cleavable bond selected from a disulfide, a diselenide, an anhydride, an ester (including a boronate ester and a phosphate ester), an amide, an imine, an acetal, an urea, a thiourea, a hydrazone, an ether, a silyl ether, an oxyme, a boronic acid, a nitro, and an azo. Each possibility represents a separate embodiment. In currently preferred embodiments, the stimulus-responsive linker comprises a single cleavable bond at its center. In additional embodiments, the stimulus is induced by a redox agent comprising a reducing agent selected from the group consisting of dithiothreitol (DTT), thiol, glutathione. NADPH, and metal complexes. Each possibility represents a separate embodiment. In other embodiments, the stimulus is induced by a redox agent comprising an oxidizing agent comprising a peroxide. In further embodiments, the stimulus is induced by an activating enzyme. In particular embodiments, the stimulus is induced by an activating enzyme selected from the group consisting of an amidase, an esterase, and an urease. Each possibility represents a separate embodiment.

In various embodiments, the stimulus-responsive linker is covalently bound at its termini to the hydrophilic segment by a group selected from the group consisting of —Z—, —X¹—Z—X²—, —Z¹—X¹—Z²—X²—, wherein Z, Z¹, and Z² are each independently selected from C₁-C₁₀ alkylene, C₂-C₁₀ alkenylene, C₂-C₁₀ alkynylene, and arylene; X, X¹, and X² are each independently selected from O, S and NH; —O—; —S—; —NH—; —C(═O)—; —C(═O)—O—; —O—C(═O)—O—; —C(═O)—NH—; —NH—C(═O)—NH—; —NH—C(═O)—O—; —S(═O)—; —S(═O)—O—; —PO(═O)—O—; triazolylene, and any combination thereof. Each possibility represents a separate embodiment.

According to the principles of the present invention, the two di-block copolymers having substantially the same hydrophilic to hydrophobic ratio as the amphiphilic tri-block copolymer remain in micellar form thereby retaining substantially all of the cargo encapsulated within the self-assembled tri-block copolymer micelles or covalently bound to the self-assembled tri-block copolymer micelles. In currently preferred embodiments, the amphiphilic tri-block copolymer is symmetric and the stimulus-responsive linker undergoes cleavage upon a stimulus at its center thereby forming two di-block copolymers which are identical.

In other embodiments, the hydrophobic segments comprise hydrophobic dendrons. In some embodiments, the hydrophobic dendrons comprise between 0 to 5 generations. In other embodiments, the hydrophobic dendrons comprise between 0 to 3 generations. In further embodiments, the hydrophobic dendrons are generation 0 (G0) dendrons. In other embodiments, the hydrophobic dendrons are generation 1 (G1) dendrons. In yet other embodiments, the hydrophobic dendrons are generation 2 (G2) dendrons. In particular embodiments, the hydrophobic dendrons are generation 3 (G3) dendrons.

In certain embodiments, each generation of the hydrophobic dendron comprises a linear or branched C₁-C₂₀ alkylene, C₂-C₂₀ alkenylene, C₂-C₂₀ alkynylene or arylene moiety which is substituted at each end with a group selected from the group consisting of —O—, —S—, —NH—, —C(═O)—, —C(═O)—O—, —O—C(═O)—O—, —C(═O)—NH—, —NH—C(═O)—NH—, —NH—C(═O)—O—, —S(═O)—, —S(═O)—O—, —PO(═O)—O—, and any combination thereof. Each possibility represents a separate embodiment.

In various embodiments, each generation of the dendron is derived from a compound having the following structure HX—Z—XH or HX—Z—CO₂H, wherein X is independently at each occurrence NH, S or O, and Z is selected from C₁-C₁₀ alkylene, C₂-C₁₀ alkenylene, C₂-C₁₀ alkynylene, and arylene. Each possibility represents a separate embodiment. In other embodiments, each generation of the dendron is derived from a compound selected from the group consisting of HX—CH₂—CH₂—XH, HX—(CH₂)₁₋₃—CO₂H, and HX—CH₂—CH(XH)—CH₂—XH wherein X is independently at each occurrence NH, S or O. Each possibility represents a separate embodiment. In particular embodiments, each generation of the dendron is derived from a compound selected from the group consisting of HS—CH₂—CH₂—OH, HS—(CH₂)₁₋₃—CO₂H and HS—CH₂—CH(OH)—CH₂—OH. Each possibility represents a separate embodiment.

In additional embodiments, the protein cleavable site comprises an enzymatically cleavable site. In further embodiments, the enzymatically cleavable site comprises a functional group selected from the group consisting of an ester, an amide, a carbamate, a carbonate, a urea, a sulfate, an amidine, an ether, a phosphate, a phosphoamide, a sulfamate, a nitro, an azo, and a trithionate. Each possibility represents a separate embodiment. In specific embodiments, the enzymatically cleavable site comprises a functional group represented by the structure of —O—C(O)—R′, —C(O)—OR′—NH—C(O)—R′ or —C(O)—NHR′ wherein R′ is C₁-C₁₂ alkyl or an aryl. Each possibility represents a separate embodiment.

In certain embodiments, the enzymatically cleavable site is cleavable by an amidase. In other embodiments, the enzymatically cleavable site is cleavable by an esterase. In yet other embodiments, the enzymatically cleavable site is cleavable by an urease.

In some embodiments, the protein is a transport protein. In a particular embodiment, the transport protein is a serum albumin.

According to the principles of the present invention, the addition of an activating enzyme disassembles the di-block copolymer micelles thereby releasing the cargo encapsulated therein or attached thereto.

In further embodiments, where the hydrophobic segments comprise hydrophobic dendrons, the enzymatically cleavable site is present at one or more of the terminal repeating units (i.e., terminal generations) of the hydrophobic dendrons, and/or in intermediary generations of the dendrons. Each possibility represents a separate embodiment.

Currently preferred self-assembled amphiphilic tri-block copolymers in accordance with the principles of the present invention are represented by the structure depicted in FIG. 2B.

According to another aspect, there is provided a method of delivering a cargo to a target site, the method comprising the steps of:
(i) providing a delivery system in micellar form comprising a cargo encapsulated within the micelles or covalently linked to micelles and a self-assembled amphiphilic tri-block copolymer comprising two substantially identical hydrophobic segments each comprising at least one protein cleavable site and a hydrophilic segment therebetween, wherein the hydrophilic segment comprises a central stimulus-responsive linker which undergoes cleavage upon a stimulus thereby forming two di-block copolymers having substantially the same hydrophilic to hydrophobic ratio as the amphiphilic tri-block copolymer;
(ii) applying a stimulus to induce cleavage of the linker thereby forming two di-block copolymers having substantially the same hydrophilic to hydrophobic ratio as the amphiphilic tri-block copolymer; and
(iii) contacting the di-block copolymers with a protein to induce cleavage of the protein cleavable site, thereby disassembling the micelles and releasing the cargo at the target site.

In various embodiments, the present invention provides a method for the preparation a tri-block copolymer amphiphile suitable as a delivery system according to the principles of the present invention, the method comprising the steps of:
(i) polymerizing a plurality of hydrophilic monomers at both termini of an activated stimulus-responsive linker to form a hydrophilic segment comprising a central stimulus-responsive linker;
(ii) functionalizing the termini of the hydrophilic segment; and
(iii) conjugating the functionalized hydrophilic segment with hydrophobic segments comprising at least one protein cleavable site.

In alternative embodiments, the present invention provides a method for the preparation a tri-block copolymer amphiphile suitable as a delivery system according to the principles of the present invention, the method comprising the steps of:
(i) reacting a stimulus-responsive linker activated at both termini with a hydrophilic polymer to form a hydrophilic segment comprising a central stimulus-responsive linker;
(ii) functionalizing the termini of the hydrophilic segment; and
(iii) conjugating the functionalized hydrophilic segment with hydrophobic segments comprising at least one protein cleavable site.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows a schematic representation of splittable TBC amphiphiles and their self-assembly into stable flower-like micelles. Activation of the trigger at the center of the hydrophilic block leads to the splitting of the TBC into two amphiphilic DBCs, which due to the increase in unimer-micelle exchange rate can be enzymatically degraded into hydrophilic polymers, leading to complete disassembly and release of encapsulated cargo. The amphiphiles are fluorescently labeled and show red-shifted emission at their assembled state due to close packing of the dyes.

FIGS. 2A-2B show the enzyme-responsive TBCs with a cleavable linker according to certain embodiments of the present invention. FIG. 2A shows a synthetic pathway for the preparation of TBCs with a redox- or UV-responsive linker at the center of the protected hydrophilic A block and the last two steps of conjugating the hydrophobic B blocks and deprotection of the hydrophilic A block. FIG. 2B shows the general structure of the TBC amphiphiles and the two different types of implemented cleavable linkers.

FIGS. 3A-3C show the structures of the different linkers used. FIG. 3A shows the structure of a redox-responsive initiator. FIG. 3B shows the structure of a photo-responsive initiator. FIG. 3C shows the structure of a non-responsive initiator.

FIGS. 4A-4E show the stimuli-induced transition from TBC to DBC amphiphiles. FIG. 4A shows a schematic representation of stimuli-induced splitting of the hydrophilic blocks in their middle, by DTT or UV light causing the transition from TBC to DBC amphiphiles. FIG. 4B shows overlays of HPLC chromatograms (taken at 420 nm) of SS-TBC before (SS-TBC) and after (SH-DBC) treatment with DTT. FIG. 4C shows overlays of HPLC chromatograms (taken at 420 nm) of DMNB-TBC before (DMNB-TBC) and after (US-DBC) UV irradiation. FIG. 4D shows DLS measurements before and after activation of DMNB-TBC (top) and SS-TBC (bottom); ([TBC]=80 μM. [DTT]=20 mM). FIG. 4E shows SEC traces of tert-butyl protected DMNB-TBC (DMNB-TBC*, top) and tert-butyl protected SS-TBC (SS-TBC*, bottom) before (DMNB-TBC or SS-TBC) and after (UV-DBC or SH-DBC) splitting upon UV irradiation or the addition of DTT (20 mM), respectively; ([TBC*]=10 mg/mL).

FIGS. 5A-5D show TEM images of micellar solution of FIG. 5A SS-TBC; FIG. 5B DMNB-TBC; FIG. 5C SH-DBC; and FIG. 5D UV-DBC.

FIGS. 6A-6F show enzymatic degradation and disassembly of the micelles before and after splitting. FIGS. 6A-6B show enzymatic degradation profiles as obtained by HPLC (solid lines) and fluorescence spectroscopy (dashed lines). FIGS. 6C-6D show DLS measurements after 8 hours of incubation with PBS (solid lines) or PLE (dashed lines).

FIGS. 6E-6F show Nile red release experiments after the addition of PBS (solid lines) or PLE (dashed lines). * Addition of DTT or PBS. #Addition of PLE or PBS. [TBC]=80 μM, [PLE]=0.1 μM. λ_{Ex 7-DEAC}=420 nm. [Nile red]=1.5 UM, λ_{Ex Nile red}=550 nm.

FIGS. 7A-7B show enzyme responsiveness of C7-TBC after treatment with DTT (C7-TBC+DTT), exposure to UV light (C7-TBC+UV) or PBS (C7-TBC). Enzymatic degradation profiles as obtained by FIG. 7A HPLC and FIG. 7B fluorescence spectroscopy.

FIG. 8 shows DLS measurements of C7-TBC after 8 hours incubation upon the addition of PBS (“C7-TBC”) or PLE, in addition to prior treatment with DTT (“C7-TBC+DTT+PLE”), exposure to UV radiation (“C7-TBC+UV+PLE”) or PBS (“C7-TBC+PLE”). [TBC]=80 μM. [PLE]=0.1 μM, [DTT]=20 mM.

FIGS. 9A-9B show micelle destabilization by BSA. Unimer/micelle fluorescence intensities ratio (480 nm/560 nm) over time upon the addition of BSA into micellar solutions of FIG. 9A SS-TBC (solid line) and SH-DBC (dashed line); and FIG. 9B DMNB-TBC (solid line) and UV-DBC (dashed lines). [TBC]=80 μM. [BSA]=5.5 mg/mL, λ_{Ex}=420 nm.

FIGS. 10A-10B show unimer/micelle fluorescence intensities ratio (480 nm/560 nm) over time for FIG. 10A SS-TBC and SH-DBC; and FIG. 10B DMNB-TBC and UV-DBC in PBS; [TBC]=72 μM, λ_{Ex}=420 nm.

FIGS. 11A-11C show the dual-activation mechanism using two enzymes. FIG. 11A shows a schematic illustration of a multi-responsive amphiphile with central esterase cleavable sites (“2”) and amidase cleavable hydrophobic end-groups (“1”). FIG. 11B shows DLS data for the assembled structures. FIG. 11C shows HPLC degradation data after two hours of incubation with each of the activating enzymes and their mixture.

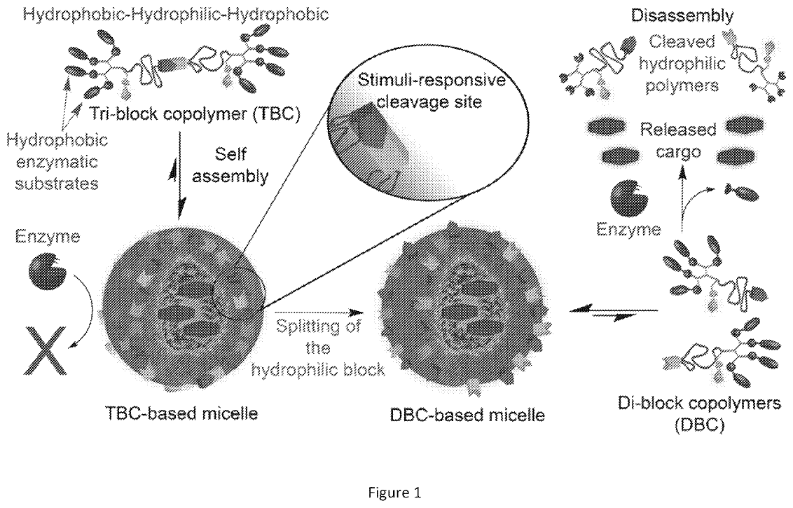

### DETAILED DESCRIPTION

The present invention provides delivery systems useful for releasing a cargo encapsulated therein or attached thereto at a target site of interest. The delivery systems comprise stable enzyme-responsive micelles whereby the enzymatic degradation of the micelles can be enhanced on demand. The control over the response to an activating enzyme is achieved by stimuli-induced splitting of tri-block amphiphiles into two di-block amphiphiles, which have substantially the same hydrophilic-lipophilic balance as the parent tri-block amphiphile. This architectural transition dramatically affects the micelle-unimer equilibrium and increases the sensitivity of the micelles towards enzymatic degradation and cargo release.

Disclosed therein for the first time is a modular approach for designing stable enzyme-responsive micelles, whose enzymatic degradation can be enhanced by applying an external stimulus. The micelles are composed of self-assembled tri-block copolymer amphiphiles that are highly stable and only become susceptible to enzymatic disassembly and cargo release when activated by the stimulus. The delivery of a cargo encapsuled within the micelles or attached to the micelles is therefore highly selective and efficient thereby overcoming the stability-degradability barrier for enzyme-responsive nanocarriers. The synthesis of the tri-block copolymer amphiphiles can be readily implemented to a variety of linkers, hydrophilic and hydrophobic moieties.

The tri-block copolymer amphiphiles of the present invention feature a central linker located at the middle of a central hydrophilic segment between two hydrophobic segments. The central linker contains a bond which is cleavable upon activation by external stimuli, for example a stimuli induced by a change in temperature, pH, light, and electric field or the addition of a redox agent or an activating enzyme. Advantageously, locating the stimuli-responsive linker at the center of the hydrophilic segment facilitates the accessibility of the linker to a degrading enzyme as compared to a stimuli-responsive linker located between a hydrophilic and hydrophobic segments.

Thus, provided herein is a delivery system in micellar form comprising a self-assembled amphiphilic tri-block copolymer. The terms “micelles” and/or “micellar form” used herein interchangeably refer to nanosized spherical, flower-like, ellipsoid, cylindrical, or unilamellar structures that are formed by self-assembly of components having hydrophobic and hydrophilic segments. The micelles typically have an average particle size of less than about 100 nm, preferably about 50 nm or lower, more preferably about 5 nm to 50 nm, and most preferably about 5 nm to 20 nm, including each value within the specified ranges. In some currently preferred embodiments, the micelles are in the form of flower-like micelles.

According to some aspects and embodiments, the tri-block copolymers have the following structure: B-A-B, where B is a hydrophobic segment comprising at least one protein cleavable site and A is a hydrophilic segment comprising a central stimulus-responsive linker which is designed to undergo cleavage upon application of a stimulus to result in two di-block copolymers having the following structure A′-B and substantially the same hydrophilic to hydrophobic ratio as the B-A-B tri-block copolymer.

### 1. Hydrophilic Segment:

In some aspects and embodiments, the hydrophilic segment comprises a hydrophilic polymer. Suitable hydrophilic polymers include, but are not limited to, polyacrylic acid, poly(2-hydroxyethyl acrylate), polyethylene glycol (PEG), and poly(oligo-ethylene glycol acrylate). Each possibility represents a separate embodiment. Additional hydrophilic polymers include, but are not limited to, polyacrylamides, polymethyl oxazoline, polyethyl oxazoline, polysarcosine, polypeptides, polypeptoids, hydrophilic polymethacrylates, polyamines, hydrophilic nylons, polyvinyl alcohol, hydrophilic proteins and polycarbohydrates. Each possibility represents a separate embodiment. In particular embodiments, the hydrophilic segment has a molecular weight of about 0.5 to about 100 kDa, including each value within the specified range. In one embodiment, the hydrophilic segment does not contain PEG or derivative thereof. In other embodiments, the hydrophilic segment does not contain a peptide or a polypeptide.

The hydrophilic segment is chemically bound at both termini to the hydrophobic segments. Typical groups in said chemical bonds include, but are not limited to, —Z—, —X¹—Z—X²—, —Z¹-X₁—Z²—Z²—, wherein Z, Z¹, and Z² are each independently selected from C₁-C₁₀ alkylene, C₂-C₁₀ alkenylene, C₂-C₁₀ alkynylene, and arylene; X, X¹, and X² are each independently selected from O, S and NH; —O—; —S—; —NH—; —C(═O)—; —C(═O)—O—; —O—C(═O)—O—; —C(═O)—NH—; —NH—C(═O)—NH—; —NH—C(═O)—O—; —S(═O)—; —S(═O)—O—; —PO(═O)—O—; triazolylene, and any combination thereof. Each possibility represents a separate embodiment.

According to the principles of the present invention, the hydrophilic segment comprises a stimulus-responsive linker chemically bound at both termini to the hydrophilic polymer. Typically, the same chemical bonds that form the conjugation of the hydrophilic polymer to the hydrophobic segments as detailed above, can also be used to conjugate the linker at both ends to the hydrophilic polymer.

In some aspects and embodiments, the hydrophilic segment has the following structure: A_{1/2}-L-A_{1/2}, where A_{1/2} represent the termini of the hydrophilic polymer and L is the stimulus-responsive linker. Typically, the stimulus-responsive linker comprises a single cleavable bond, for example a disulfide, a diselenide, an anhydride, an ester (including a boronate ester and a phosphate ester), an amide, an imine, an acetal, an urea, a thiourea, a hydrazone, an ether, a silyl ether, an oxyme, a boronic acid, a nitro, or an azo at its center. Each possibility represents a separate embodiment. In various embodiments, the stimulus-responsive linker comprises two cleavable bonds as detailed above which are separated by a spacer. The cleavable bond(s) can be cleaved by a chemically-induced stimulus such as, but not limited to, the addition of a redox agent or an activating enzyme. Each possibility represents a separate embodiment. Suitable redox agents include, but are not limited to, reducing agents, for example, dithiothreitol (DTT), thiol, glutathione, NADPH, and metal complexes. Each possibility represents a separate embodiment. Additional redox agents include, but are not limited to, oxidizing agents, for example hydrogen peroxide. Suitable activating enzymes include, but are not limited to, esterases, ureases, amidases, uricases, creatininases, lipases, cellulases, amylases, pectinases, acylases, catalases, proteinase-K, nitro and azo reductases, cathepsin, and matrix metalloproteinase. Each possibility represents a separate embodiment. Currently preferred are esterases, ureases or amidases including, but not limited to, aryl-acylamidase, aminoacylase, alkylamidase, phthalyl amidase, carboxylesterase, arylesterase, and acetylesterase. Each possibility represents a separate embodiment.

In other aspects and embodiments, the cleavable bond can be cleaved by a physically-induced stimulus such as, but not limited to, a change in at least one of temperature, pH, light (UV light or near infrared light), ultrasound, electric field, and electromagnetic radiation. Each possibility represents a separate embodiment. As will be appreciated to those skilled in the art, disulfide and diselenide bonds in the linker may be cleaved by the addition of reducing agents such as dithiothreitol (DTT) and thiols; and ester and amide bonds can be cleaved by enzymatic cleavages. Thus, the stimulus that is applied is chosen to accord with the cleavable bond in the stimulus-responsive linker.

The stimulus-responsive linker is therefore designed to contain a chemical bond or a plurality of chemical bonds that are cleaved upon application of one or more of the aforementioned stimuli. In currently preferred embodiments, the stimulus-responsive linker comprises a single cleavable bond which is located at the center of the linker and/or at the center of the hydrophilic segment thereby resulting in the following structures: A_{1/2}-L_{1/2}-L_{1/2}-A_{1/2}, where A_{1/2}-L_{1/2} is A′ which forms the hydrophilic segment of the di-block amphiphiles.

According to the principles of the present invention, the two di-block copolymers are characterized by having substantially the same hydrophilic to hydrophobic ratio as the amphiphilic tri-block copolymer thereby maintaining the micellar architecture/configuration.

As used herein, the term “hydrophilic to hydrophobic ratio” refers to the ratio of the hydrophilic units to the hydrophobic units. In other embodiments, the term “hydrophilic to hydrophobic ratio” refers to the hydrophilic to lipophilic balance (HLB) which represents the degree of affinity to water and oil. Specifically, wherein the HLB is close to zero, the amphiphile is considered highly hydrophobic, and wherein the HLB is close 20, the amphiphile is considered highly hydrophilic. The HLB value may be determined by any method known in the art, for example the Atlas method, the Griffin method, the Davis method, or the Kawakami method. Each possibility represents a separate embodiment. As used herein, the term “substantially” refers to a deviation that is of not more than ±10% of the hydrophilic to hydrophobic ratio or the HLB value.

According to the principles of the present invention, by maintaining substantially the same hydrophilic to hydrophobic ratio of the di-block amphiphiles as that of the tri-block amphiphiles, the di-block amphiphiles remain in micellar form thereby retaining substantially all of the cargo within the self-assembled tri-block copolymer micelles. In currently preferred embodiments, the amphiphilic tri-block copolymer is symmetric and the stimulus-responsive linker undergoes cleavage upon a stimulus at its center thereby forming two di-block copolymers which are identical.

### 2. Hydrophobic Segments:

In some aspects and embodiments, the hydrophobic segments comprise hydrophobic dendrons. A “dendron” as used herein is a hyper-branched monodisperse organic molecule defined by a tree-like or generational structure. In general, dendrons possess three distinguishing architectural features: a linker moiety; an interior area containing generations with radial connectivity to the linker moiety; and a surface region (peripheral region) of terminal moieties. According to certain embodiments, each generation of the hydrophobic dendron comprises a linear or branched C₁-C₂₀ alkylene, C₂-C₂₀ alkenylene, C₂-C₂₀ alkynylene or arylene moiety which is substituted at each end with a group selected from the group consisting of —O—, —S—, —NH—, —C(═O)—, —C(═O)—O—, —O—C(═O)—O—, —C(═O)—NH—, —NH—C(═O)—NH—, —NH—C(═O)—O—, —S(═O)—, —S(═O)—O—, —PO(═O)—O—, and any combination thereof. Each possibility represents a separate embodiment.

In certain aspects and embodiments, each generation is derived from a compound having a structure represented by the formulae HX—Z—XH or HX—Z—CO₂H, wherein X is independently at each occurrence NH, S or O, and Z is selected from C₁-C₁₀ alkylene, C₂-C₁₀ alkenylene, C₂-C₁₀ alkynylene, and arylene. Each possibility represents a separate embodiment. According to other embodiments, each generation of the dendron is derived from a compound selected from the group consisting of HX—CH₂—CH₂—XH, HX—(CH₂)₁₋₃—CO₂H, and HX—CH₂—CH(XH)—CH₂—XH wherein X is independently at each occurrence NH, S or O. Each possibility represents a separate embodiment. In one currently preferred embodiment, each generation of the dendron is derived from a compound selected from the group consisting of HS—CH₂—CH₂—OH, HS—(CH₂)₁₋₃—CO₂H and HS—CH₂—CH(OH)—CH₂—OH. Each possibility represents a separate embodiment.

The hydrophobic dendron of the present invention comprises a preferred number of generations in the range of 0 to 5, more preferably 0 to 3, including each integer within the specified ranges. In one embodiment, the hydrophobic dendron is a generation 0 (G0) dendron. In another embodiment, the hydrophobic dendron is a generation 1 (G1) dendron. In yet another embodiment, the hydrophobic dendron is a generation 2 (G2) dendron. In other embodiments, the hydrophobic dendron is a generation 3 (G3) dendron.

In various embodiments, the dendron comprises a repeating unit selected from the group consisting of:

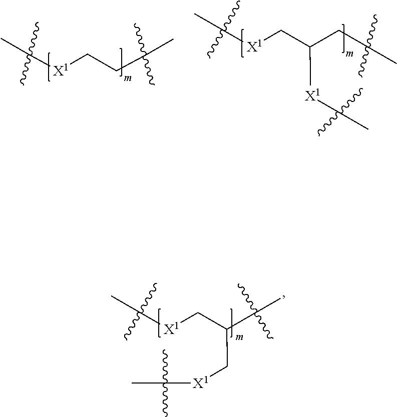

wherein X¹ is independently, at each occurrence, selected from the group consisting of O, S and NH; and m is an integer from 1 to 15, including each integer within the specified range.

According to the principles of the present invention, the hydrophobic segment comprises at least one protein cleavable site, for example an enzymatically cleavable site. A “protein cleavable site” as used herein refers to a region of a compound that is chemically altered in the presence of one or more proteins. In some embodiments, a “protein cleavable site” refers to a region of a compound that is totally or partially cleaved by one or more proteins, for example a region that is enzymatically cleavable. Enzymatically cleavable sites typically include a functional group such as, but not limited to, an ester, an amide, a carbamate, a carbonate, a urea, a sulfate, an amidine, an ether, a phosphate, a phosphoamide, a sulfamate, a nitro, an azo, and a trithionate. Each possibility represents a separate embodiment. Functional groups that can be cleaved by enzymes include, for example —O—C(O)—R′, —C(O)—OR′—NH—C(O)—R′ or —C(O)—NHR′ wherein R′ is C₁-C₁₂ alkyl or an aryl. Each possibility represents a separate embodiment.

It will be appreciated to one skilled in the art that an amide bond is enzymatically cleavable by an amidase. Suitable amidases that can cleave an amide bond include, but are not limited to, aryl-acylamidase, aminoacylase, alkylamidase, and phthalyl amidase. Each possibility represents a separate embodiment. Where an ester bond is present in the hydrophobic segment, it can be cleaved by an esterase. Suitable esterases that can cleave an ester bond include, but are not limited to, carboxylesterase, arylesterase, and acetylesterase. Each possibility represents a separate embodiment. Where an urea bond is present in the hydrophobic segment, it can be cleaved by an urease. Additionally, proteins other than enzymes can be used to chemically alter the sites in the hydrophobic segments thereby disassembling the micelles and releasing the cargo. These include, for example, transport proteins such as, but not limited to, Serum Albumin (e.g., BSA or HSA) and Keyhole Limpet Hemocyanin (KLH). Each possibility represents a separate embodiment.

Where hydrophobic segments comprise hydrophobic dendrons, the enzymatically cleavable site may be present at one or more of the terminal repeating units (i.e., terminal generations) of the hydrophobic dendron, and/or in intermediary generations of the dendron. The enzymatically cleavable hydrophobic end group may be present only at the terminal repeating units of the hydrophobic dendron (i.e., the enzymatically cleavable hydrophobic end group is not present in intermediary generations of the dendron) or it may be present only at the intermediary generations of the dendron (i.e., the enzymatically cleavable hydrophobic end group is not present in the terminal repeating units of the hydrophobic dendron). Each possibility represents a separate embodiment.

The term “alkyl” used herein alone or as part of another group denotes a saturated aliphatic hydrocarbon, including straight-chain and branched-chain alkyl groups. In one embodiment, the alkyl group has 1-12 carbons designated here as C₁-C₁₂ alkyl. In another embodiment, the alkyl group has 1-4 carbons designated here as C₁-C₄ alkyl. Examples of alkyl groups include, but are not limited to, methyl, ethyl, propyl, isopropyl, n-butyl, isobutyl, sec-butyl, t-butyl, and the like.

The term “alkylene” used herein alone or as part of another group denotes a bivalent radical which is bonded at two positions connecting together two separate additional groups (e.g., CH₂). Examples of alkylene groups include, but are not limited to —(CH₂)—, —(CH₂)₂—, —(CH₂)₃—, —(CH₂)₄—, etc.

The term “alkenylene” used herein alone or as part of another group denotes a bivalent radical containing at least one double bond, which is bonded at two positions connecting together two separate additional groups (e.g., —CH═CH—).

The term “alkynylene” used herein alone or as part of another group denotes a bivalent radical containing at least one triple bond, which is bonded at two positions connecting together two separate additional groups (e.g., —C≡C—).

The term “aryl” used herein alone or as part of another groups denotes an aromatic ring system containing from 4-14 ring carbon atoms. The aryl ring can be a monocyclic, bicyclic, tricyclic and the like. Non-limiting examples of aryl groups are phenyl, naphthyl including 1-naphthyl and 2-naphthyl, and the like.

The term “arylene” denotes a bivalent radical of aryl, which is bonded at two positions connecting together two separate additional groups (e.g., —C₆H₄—).

Each of the alkyl, alkylene, alkenylene, alkynylene, aryl, and arylene can be substituted by one or more of the following substituents methyl, ethyl, propyl, isopropyl, butyl, tert-butyl, halogen, haloalkyl, hydroxy, alkoxy, carbonyl, amido, alkylamido, dialkylamido, nitro, cyano, amino, alkylamino, dialkylamino, carboxyl, thio, and thioalkyl. Each possibility represents a separate embodiment.

All stereoisomers, optical and geometrical isomers of the compounds of the instant invention are contemplated, either in admixture or in pure or substantially pure form. The compounds of the present invention can have asymmetric centers at any of the atoms. Consequently, the compounds can exist in enantiomeric or diastereomeric forms or in mixtures thereof. The present invention contemplates the use of any racemates (i.e., mixtures containing equal amounts of each enantiomers), enantiomerically enriched mixtures (i.e., mixtures enriched in one enantiomer), pure enantiomers or diastereomers, or any mixtures thereof. The chiral centers can be designated as R or S or R,S or d,D, l,L or d,l, D,L. In addition, several of the compounds of the invention contain one or more double bonds. The present invention intends to encompass all structural and geometrical isomers including cis, trans, E and Z isomers, independently at each occurrence. Any salt form with both basic and acid addition salts is also contemplated within the scope of the present invention.

The tri-block copolymers of the present invention can be prepared by the following method. First, the hydrophilic segment is formed by polymerizing a plurality of hydrophilic monomers at both ends of an activated stimulus-responsive linker. The polymerization can be performed, for example, by an atom transfer radical polymerization. Alternatively, an activated stimulus-responsive linker may be reacted at both ends with a pre-formed hydrophilic polymer to form the hydrophilic segment. Second, the hydrophilic segment is functionalized at its the terminal ends to afford its reaction with the hydrophobic segments that contain the protein cleavable sites. Functionalization can be performed, for example by azidation of the hydrophilic segment. Finally, the conjugation of the functionalized hydrophilic segment with the hydrophobic segments can be performed, for example, by a click reaction.

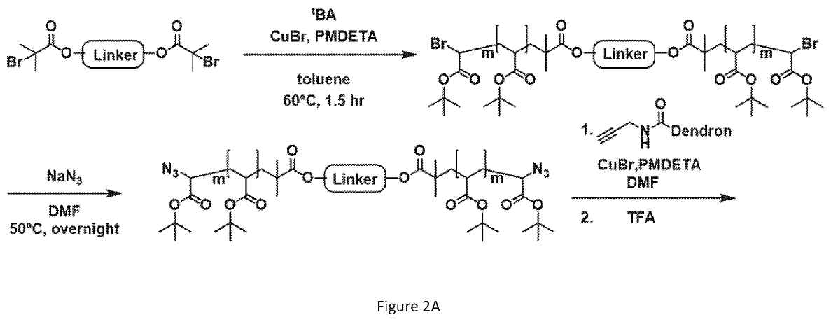

In one embodiment, the tri-block copolymer is represented by the structure depicted in FIG. 2B.

### 3. Cargo Release and Uses:

In some aspects and embodiments, the delivery system disclosed herein comprises a cargo encapsulated within the micelles. In other aspects and embodiments, the delivery system disclosed herein comprises a cargo covalently attached to the micelles. Suitable cargo includes, but is not limited to, pharmaceutical agents, agrochemical agents, cosmetic agents, imaging agents, and/or diagnostic agents. Each possibility represents a separate embodiment. According to the principles disclosed herein, the cargo is released from the micelles on demand upon disassembly of the micelles.

Pharmaceutical agents include, but are not limited to, drugs which may be small molecules or biologics. Non-limiting examples of drugs include chemotherapeutic agents, anti-proliferative agents, anti-cancer agents, inhibitors, receptor agonists, receptor antagonists, co-factors, anti-inflammatory drugs (steroidal and non-steroidal), antipsychotic agents, analgesics, anti-thrombogenic agents, anti-platelet agents, anticoagulants, anti-diabetics, statins, toxins, antimicrobial agents, anti-histamines, metabolites, anti-metabolic agents, vasoactive agents, vasodilator agents, cardiovascular agents, antioxidants, phospholipids, and heparins. Each possibility represents a separate embodiment. Pharmaceutical agents also include peptides, polypeptides, hormones, polymers, amino acids, oligonucleotides, nucleic acids, genes, growth factors, enzymes, co-factors, antisense molecules, antibodies, antigens, vitamins, immunoglobulins, cytokines, prostaglandins, vitamins, toxins and the like, as well as organisms such as bacteria, viruses, fungi and the like. Each possibility represents a separate embodiment.

Agrochemical agents include, but are not limited to, a pesticide, an insecticide, a herbicide, a fungicide, an acaricide, an algicide, an antimicrobial agent, biopesticide, a biocide, a disinfectant, a fumigant, an insect growth regulator, a plant growth regulator, a miticide, a microbial pesticide, a molluscide, a nematicide, an ovicide, a pheromone, a repellent, a rodenticide, a defoliant, a dessicant, a termiticide, a piscicide, avicide, rodenticide, bactericide, insect repellent, an auxin, a cytokinin, a gametocide, a gibberellin, a growth inhibitor, a growth stimulator and any combination thereof. Each possibility represents a separate embodiment.

Cosmetic agents include, but are not limited to, hyaluronic acid, vitamins and vitamin derivatives such as, for example vitamin A, vitamin B, vitamin D, vitamin E, vitamin K and derivatives thereof including, for example, a tocopherol; various plant extracts such as, for example Aloe vera, Aloe barbadensis, castor oil, Citrus limonium, Citrus paradisi, Citrus sinensis, Elaesis guineensis, etc. sunscreens and tanning agents and the like. Each possibility represents a separate embodiment.

Imaging and/or diagnostic agents include, but are not limited to, labeling compounds or moieties such as chromophores, fluorescent compounds or moieties, phosphorescent compounds or moieties, contrast agents, radioactive agents, magnetic compounds or moieties (e.g., diamagnetic, paramagnetic and ferromagnetic materials), heavy metal clusters and the like. Each possibility represents a separate embodiment.

The cargo is typically present in the self-assembled amphiphiles in amounts sufficient so as to exert its beneficial effect once released from the micelles.

According to the principles of the present invention, cargo delivery occurs at two stages. First, a stimulus to the tri-block amphiphiles is applied to induce cleavage of the linker in the hydrophilic middle segment thereby forming two di-block copolymers having substantially the same hydrophilic to hydrophobic ratio as the amphiphilic tri-block copolymer. While the stimulus results in the transition from a tri-block copolymer to diblock copolymers, it maintains the architecture/configuration of the amphiphiles in micellar form thereby maintaining the cargo within the micelles. Then, the di-block copolymers are contacted with a protein to induce cleavage of the protein cleavable site in the hydrophobic segments, thereby disassembling the micelles and releasing the cargo at the target site.

As used herein, the term “contacting” refers to bringing in contact with the self-assembled amphiphiles of the present invention. Contacting can be accomplished to cells or tissue cultures, or to living organisms, for example humans. In one embodiment, the present invention encompasses contacting the self-assembled amphiphiles of the present invention within a human subject. In other embodiments, the term “contacting” may be ex-vivo on a surface, on a device, in cell/tissue culture dish, in food and water.

The delivery system of the present invention can further be provided in the form of a kit whereby one compartment comprises the tri-block copolymer amphiphile and at least one other compartment comprises the protein capable of cleaving the enzymatically cleavable site(s) in the hydrophobic segments so as to disassemble the micelles and release the cargo. In other embodiments, the kit further comprises another compartment that comprises a buffer, a redox agent or an activating enzyme capable of cleaving the linker in the hydrophilic segment thereby rendering the micelles more susceptible to disassembly when in contact with the protein that induces disassembly and cargo release. When the protein/enzyme is in a lyophilized form, the kit may optionally contain a sterile and physiologically acceptable reconstitution medium such as water, saline, buffered saline, and the like. Each possibility represents a separate embodiment. According to some embodiments, associated with such compartments in the kit may be various written instructions for use.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to ±10% of the reference value.

It is noted that as used herein and in the appended claims, the singular forms “a”, “an”, and “the” include plural referents unless the context clearly dictates otherwise. Thus, for example, reference to “a cleavable site” includes a plurality of such sites and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a composition having at least one of A, B, and C” would include but not be limited to compositions that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Example 1

### Synthesis of Tri-Block Amphiphiles

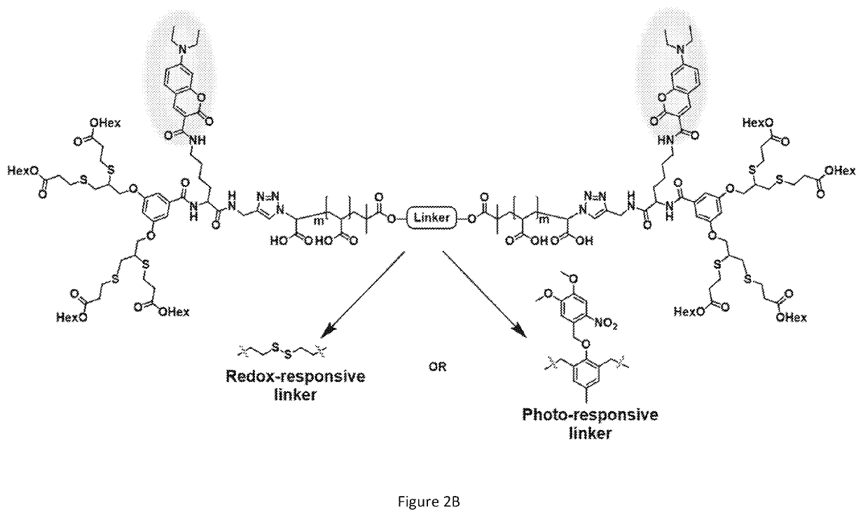

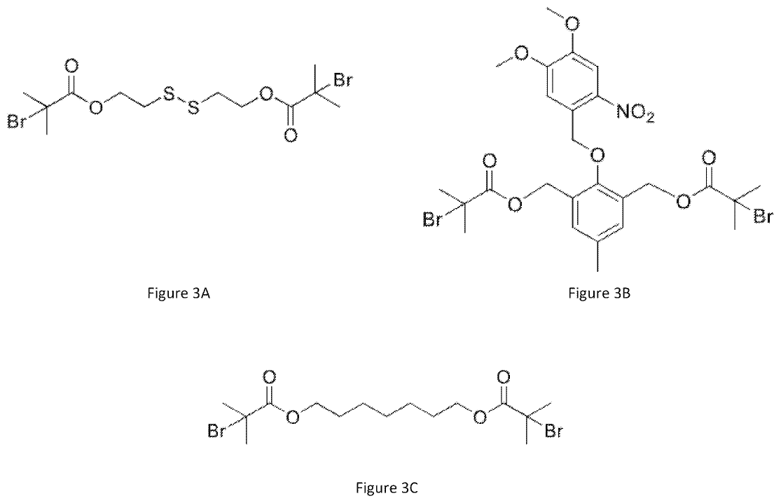

The synthesis of the TBCs was performed by the following steps. Briefly, atom transfer radical polymerization (ATRP) of tert-butyl acrylate (BA) from a bifunctional initiator that contained the splittable linker at its center was performed according to Peles-Strahl et al. Macromolecules 2019, 52 (9), 3268-3277; Davis et al. Macromolecules 2000, 33 (11), 4039-4047; and Moreno et al. Macromolecules 2020, 53 (17), 7285-7297 (FIG. 2A). Two stimuli-responsive linkers were used: (1) a redox-responsive linker that bears a disulfide bond at its center (FIG. 3A), the redox-responsive linker can be cleaved by a reducing agent such as dithiothreitol (DTT), and (2) a UV-responsive-linker that contains 4,5-dimethoxy-2-nitrobenzyl (DMNB) linked by an AB2 self-immolative spacer (FIG. 3B; Peles-Strahl et al. Macromolecules 2019, 52 (9), 3268-3277; and Amir et al., Angew. Chemie-Int. Ed. 2003, 42 (37), 4494-4499).

Post-polymerization, the terminal bromides were substituted by azides, which were conjugated by copper catalyzed azide-alkyne cycloaddition (CuAAC) with esterase-cleavable hydrophobic dendrons (Golas et al., Macromolecules 2006, 39 (19), 6451-6457; and Slor et al. Biomacromolecules 2021, 22 (3), 1197-1210). The dendrons were fluorescently labeled with 7-diethylamino-3-carboxy coumarin (7-DEAC) due to its ability to form excimers when the micelles are assembled, as indicative by a fluorescence emission maximum at 560 nm (rather than at 480 nm, the emission maximum of the free dye). This red-shifted emission of the fluorescently labeled amphiphiles in the assembled state was used to provide structural information on the micellar mesophase. At the last step of the synthesis, the tert-butyl protecting groups were removed under acidic conditions, exposing highly hydrophilic carboxylic acids of the poly(acrylic acid) (PAA) backbone (FIG. 2B).

All TBCs were obtained in high purity and characterized by NMR, HPLC, SEC, UV-VIS and fluorescence spectroscopies.

In particular, the synthesis route for the preparation of azide functionalized PtBAs is shown in Scheme 1:

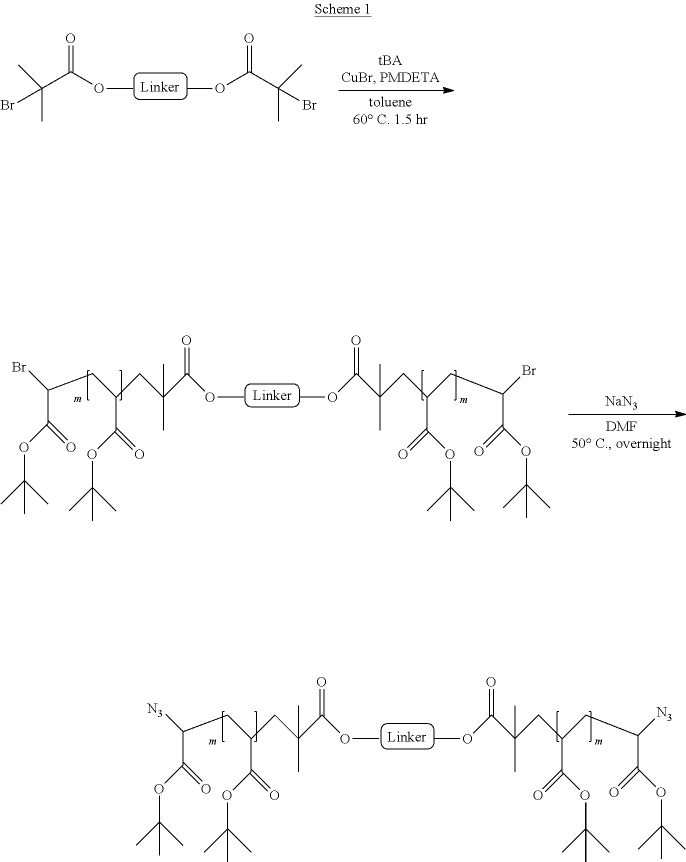

The general procedure for ATRP polymerization was performed as follows: 50 ml tert butyl acrylate (tBA) were washed with 5% NaOH aqueous solution (3×40 ml) and then with water (40 ml), dried over MgSO₄ and distilled in vacuum (˜60° C., 30 mbar). The desired initiator (1 eq.) was dissolved in 2 ml toluene in a separate vial. PMDETA (2 eq) was dissolved in tBA (50 eq.) and 0.5 ml toluene. Both vials were purged with nitrogen for 15 minutes. CuBr (2 eq) was loaded to a round bottom shlenk flask, which was evacuated and backfilled with nitrogen three times and left under nitrogen atmosphere. PMDETA and tBA mixture was added using nitrogen flushed syringe and needle and stirred with CuBr for 20 minutes at room temperature until the solution became clear and greenish. The initiator solution was then added using nitrogen flushed syringe and needle and the flask was submerged in an oil bath preheated to 60° C. and stirred for 1.5 hr. The reaction was stopped by opening the flask to air and cooling it in an ice bath. The reaction mixture was then filtered through Celite and neutral alumina, concentrated in vacuum, redissolved in 50 ml THF and precipitated into 500 ml ice cold mixture of water and MeOH (1:1 v/v). The solvents were decanted and another precipitation was performed. After decanting the water and MeOH mixture, the white residue was dissolved in DCM, dried over Na₂SO₄ and evaporated to dryness. The polymers were obtained as white solids. The degree of polymerization (DP) was determined using ¹H NMR spectroscopy by comparing the integration of the four methyl groups of the initiator (˜1.1 ppm, calibrated as 12H) to the methine (CH) of the polymer backbone (˜2.0-2.5 ppm).

The general procedure for azidation of PtBA was performed as follows: bromide functionalized PtBA (1 eq.) was dissolved in DMF (10-15 ml per 1 gr polymer), NaN₃ (20 eq.) was added and the reaction was stirred overnight at 50° C. The reaction was then cooled to room temperature and diluted with ether (150 ml) which was washed with water (3×100) ml), dried over Na₂SO₄ and evaporated to dryness. The products were obtained as white solids.

SS-PtBA-Br: Redox-responsive initiator (502 mg, 1.111 mmol), PMDETA (470) μl, 2.22 mmol), tBA (8.13 ml, 55.5 mmol) and CuBr (318 mg, 2.22 mmol) were reacted according to the general procedure. The polymer was obtained as a white solid in 79% yield (5.5 gr). ¹H NMR (400 MHZ, Chloroform-d) δ 4.30 (br s, 4H, CH₂—CH₂—O), 4.10 (m, 2H, —CH—Br), 3.03-2.80 (t, J=8.0 Hz, 4H, CH₂—CH₂—S—), 2.44-2.06 (brs, 43H, PtBA backbone —CH—CO), 2.02-1.17 (m, 503H. PtBA backbone —CH₂—CH—+—O—C(CH₃)₃), 1.13 (s, 12H, —CO—C—(CH₃)₂-PtBA). DP=45. Monomer conversion=90%. SEC (DMF+25 mM NH₄Ac): Mn=3.6 kDa, D=1.12, Expected Mn=6.3 kDa.

DMNB-PtBA-Br: Photo-responsive initiator (200 mg, 0.30 mmol). PMDETA (128 μl, 0.60 mmol), tBA (2.21 ml, 15.12 mmol) and CuBr (87 mg, 0.60 mmol) were reacted according to the general procedure. The polymer was obtained as a white solid in 73% yield (1.4 gr). ¹H NMR (400 MHZ, Chloroform-d) δ 7.75 (s, 1H, Ar—H), 7.61 (s, 1H, Ar—H), 7.24 (s, 2H, Ar—H), 5.29 (s, 2H, Ar—CH₂—O—), 5.09 (s, 4H, Ar—CH₂—O—), 4.20-4.04 (m, 2H, —CH—Br), 4.03 (s, 3H, —O—CH₃), 3.96 (s, 3H, —O—CH₃), 2.34 (s, 3H, Ar—CH₃), 2.31-2.10 (brs, 41H, PtBA backbone —CH—CO), 1.93-1.17 (m, 463H, PIBA backbone —CH₂—CH—+—O—C(CH₃)₃), 1.08 (s, 12H, —CO—C—(CH₃)₂-PtBA). DP=44, Monomer conversion=88%, SEC (DMF+25 mM NH₄Ac): Mn=3.6 kDa, D=1.11, Expected Mn=6.4 kDa.

In addition to the redox-responsive (SS-TBC) and the UV-responsive (DMNB-TBC) amphiphiles, a non-responsive TBC with a heptyl chain at its center (C7-TBC; FIG. 3C) was synthesized and used as a control. The synthesis of C7-TBC was performed as follows: 1,7-heptanediol (0.7 ml, 5.03 mmol) and Et₃N (2.1 ml, 15.10 mmol) were dissolved in DCM (30 ml) and cooled to 0° C. «-bromoisobutyryl bromide (1.9 ml, 15.10 mmol) was dissolved in DCM (10 ml) and added dropwise. The reaction was allowed to heat to room temperature and stirred for 1 hr. The organic phase was washed with a saturated NH₄Cl solution and a saturated NaHCO₃ solution (20 ml each), dried over Na₂SO₄ and the solvents were removed under reduced pressure. The product was purified by a silica column (90:10 Hex:EA, TLC plates were stained with KMnO₄). The product was obtained as a colorless oil in 79% yield (1.7 gr). ¹H NMR (400 MHZ. Chloroform-d) δ 4.15 (t, J=6.6 Hz, 4H, CH₂—CH₂—O—), 1.91 (s, 12H, —CO—C—(CH₃)₂—Br), 1.66 (m, 4H, CH₂—CH₂—CH₂—O—), 1.46-1.28 (m, 6H, O—CH₂—CH₂—(CH₂)₃—CH₂—CH₂—O). ¹³C NMR (100 MHz, Chloroform-d) δ 171.7, 65.9, 55.9, 30.7, 28.6, 28.2, 25.6. MS (m/z. [M+Na]+, ESI): calculated: 453.0, found: 453.1.

C7-PtBA-Br: C7 non-responsive initiator (275 mg, 0.61 mmol), PMDETA (271 μl, 1.22 mmol), tBA (4.70 ml, 30.5 mmol) and CuBr (184 mg, 1.22 mmol) were reacted according to the general procedure. The polymer was obtained as a white solid in 77% yield (3.3 gr). ¹H NMR (400 MHZ, Chloroform-d) δ 4.29-3.88 (m, 6H, CH₂—CH₂—O—+—CH—Br), 2.54-2.05 (brs, 49H, PtBA backbone —CH—CO), 2.03-1.18 (m, 522H, O—CH₂—(CH₂)₅—CH₂—O)+PtBA backbone —CH₂—CH—+—O—C—(CH₃)₃), 1.12 (s, 12H, —CO—C—(CH₃)₂-PtBA). DP=50, Monomer conversion=100%. SEC (DMF+25 mM NH₄Ac): Mn=4.7 kDa, D=1.14, Expected Mn=7.0 kDa.

SS-PtBA-N₃: SS-PtBA-Br (1.1 gr, 0.170 mmol) and NaN₃ (224 mg, 3.40 mmol) were reacted according to the general procedure. The product was obtained as a white solid in 94% yield (1.03 gr). ¹H NMR (400 MHZ, Chloroform-d) δ 4.29 (br s, 4H, CH₂—CH₂—O), 3.83-3.57 (m, 2H, —CH—N₃), 2.91 (br s, 4H, CH₂—CH₂—S—), 2.40-2.12 (brs, 38H, PtBA backbone —CH—CO), 2.03-1.21 (m, 486H, PtBA backbone —CH₂—CH—+—O—C(CH₃)₃), 1.14 (s, 12H, —CO—C—(CH₃)₂-PtBA). SEC (DMF+25 mM NH₄Ac): Mn=3.7 kDa, D=1.12, Expected Mn=6.3 kDa.

DMNB-PIBA-N₃: DMNB-PtBA-Br (1.1 gr, 0.172 mmol) and NaN₃ (224 mg, 3.44 mmol) were reacted according to the general procedure. The product was obtained as a white solid in 91% yield (1.0 gr). ¹H NMR (400 MHZ, Chloroform-d) δ 7.75 (s, 1H, Ar—H), 7.61 (s, 1H, Ar—H), 7.23 (s, 2H, Ar—H), 5.29 (s, 2H, Ar—CH₂—O—), 5.09 (s, 4H, Ar—CH₂—O—), 4.03 (s, 3H, —O—CH₃), 3.96 (s, 3H, —O—CH₃), 3.83-3.55 (m, 2H, —CH—N₃), 2.35 (s, 3H, Ar—CH₃), 2.30-2.09 (brs, 40H, PtBA backbone —CH—CO), 2.01-1.20 (m, 460H, PtBA backbone —CH₂—CH—+—O—C(CH₃)₃), 1.08 (s, 12H, —CO—C—(CH₃)₂-PtBA). SEC (DMF+25 mM NH₄Ac): Mn=3.7 kDa, D=1.14. Expected Mn=6.4 kDa.

C7-PIBA-N₃: C7-PtBA-Br (450 mg, 0.07 mmol) and NaN₃ (91 mg, 1.40 mmol) were reacted according to the general procedure. The product was obtained as a white solid in 91% yield (410 mg). ¹H NMR (400 MHZ, Chloroform-d) δ 4.01 (t, J=6.7 Hz, 4H, CH₂—CH₂—O—), 3.82-3.59 (m, 2H, —+—CH—N₃), 2.39-2.09 (brs, 46H, PtBA backbone —CH—CO), 1.96-1.21 (m, 555H, O—CH₂—(CH₂)₅—CH₂—O+PtBA backbone —CH₂—CH—+—O—C(CH₃)₃), 1.13 (s, 12H, —CO—C—(CH₃)₂-PIBA). SEC (DMF+25 mM NH₄Ac): Mn=4.8 kDa, D=1.15, Expected Mn=7.0 kDa.

The synthesis route for the click reaction between di-azide functionalized PtBA and esterase responsive dendron is shown in Scheme 2:

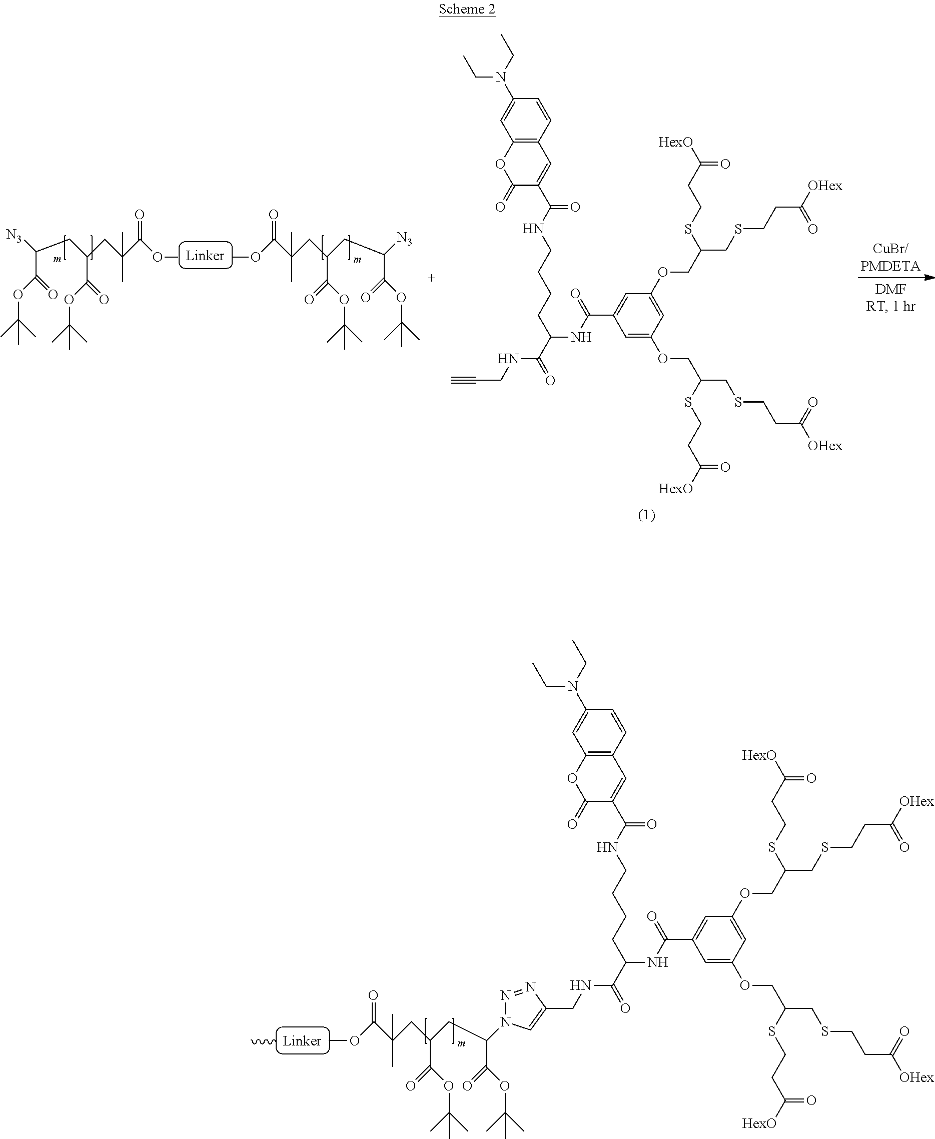

Dendron (1) was synthesized as reported in Slor et al., Biomacromolecules 2021, 22 (3), 1197-1210. The general procedure for CuAAC click reaction between di-azide functionalized PtBA and dendron was performed as follows: CuBr (3 eq. in respect to di-azide functionalized PtBA) was loaded in 4 ml glass vial, which was sealed with a rubber septum. Vial was deoxygenated with three vacuum-nitrogen cycles and backfilled with nitrogen. In a separate 4 mL vial, polymer-N₃ (1 eq.), dendron (2.6 eq.) and PMDETA (3 eq.) were dissolved in DMF (100-200 mg polymer/ml) and purged with nitrogen for 2 minutes. The mixture was added to the CuBr containing vial using nitrogen flushed syringe and needle. The vial was thoroughly vortexed until a clear green solution was obtained (approximately 30 seconds). The reaction was stirred at room temperature for 1 h, filtered through syringe filter (0.44 μm, hydrophilic PTFE) and purified using LH20 (SephadexR) size exclusion column and eluted with MeOH. Fractions that contained the product (identified by a bright yellow color) were unified and MeOH was evaporated to dryness and the product was dried on high vacuum. All polymers were obtained as bright yellow solids.

SS-PtBA-(D)-4×Hex: CuBr (7 mg, 0.048 mmol), SS-PtBA-N₃ (100 mg, 0.016 mmol), dendron (287 μl from 200 mg/ml solution in DMF, 0.041 mmol) and PMDETA (10 μl, 0.048 mmol) were reacted according to the general procedure. The product was obtained as a yellow solid in 89% yield (130 mg). SEC (DMF+25 mM NH₄Ac): Mn=5.9 kDa, D=1.20, Expected Mn=9.1 kDa.

DMNB-PtBA-(D)-4×Hex: CuBr (6 mg, 0.042 mmol), DMNB-PtBA-N₃ (90 mg, 0.014 mmol), dendron (256 μL from 200 mg/ml solution in DMF, 0.036 mmol) and PMDETA (9 μL, 0.042 mmol) were reacted according to the general procedure. The product was obtained as a yellow solid in 91% yield (130 mg). SEC (DMF+25 mM NH₄Ac): Mn=5.8 kDa, D=1.24, Expected Mn=9.2 kDa.

C7-PtBA-(D)-4×Hex: CuBr (7 mg, 0.048 mmol), C7-PtBA-N₃ (100 mg, 0.015 mmol), dendron (280 μL from 200 mg/ml solution in DMF, 0.040 mmol) and PMDETA (10 μL, 0.048 mmol) were reacted according to the general procedure. The product was obtained as a yellow solid in 88% yield (123 mg). SEC (DMF+25 mM NH₄Ac): Mn=7.0 kDa, D=1.19, Expected Mn=9.8 kDa.

The general procedure for tert-butyl deprotection of PtBA was performed as follows: Tert-butyl protection was removed by dissolving 100 mg of linker-PtBA-(D)-4×Hex in 3 ml TFA and stirring at room temperature for 1 h. TFA was removed under reduced pressure and further dried under high vacuum for 30 minutes. The product was redissolved in MeOH and further purified by LH20 SEC, to afford the final TBC amphiphiles (linker-PAA-(D)-4×Hex). Due to the treatment with TFA, the TBC amphiphiles were partially splitted into DBC. All fractions were analyzed using HPLC (420 nm), and those with sufficient purity (above 95% TBC) were collected and used for further experiments and analysis.

The synthesis route for the deprotection is shown in Scheme 3:

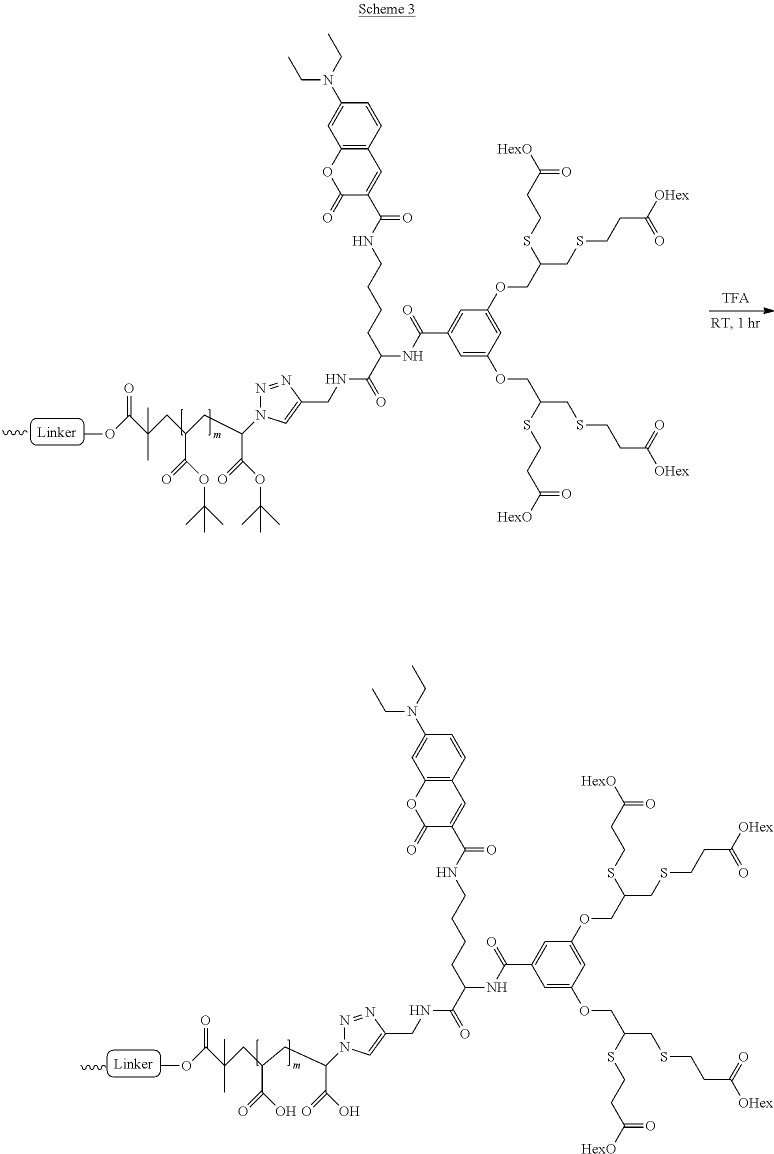

SS-PAA-(D)-4×Hex (SS-TBC): SS-PtBA-(D)-4×Hex was treated with TFA according to the general procedure. ¹³C-NMR (100 MHZ, CD₃OD): δ 179.3, 178.4, 174.7, 173.6, 164.6, 160.9, 159.0, 154.5, 149.2, 137.2, 132.7, 111.7, 110.1, 109.5, 107.7, 106.4, 97.4, 71.1, 66.0, 63.5, 55.3, 47.1, 46.1, 42.8, 40.0, 37.9, 37.2, 36.2, 36.2, 35.8, 32.6, 30.0, 29.3, 28.4, 28.2, 26.7, 25.9, 24.3, 23.6, 14.5, 13.0.

DMNB-PAA-(D)-4×Hex (DMNB-TBC): DMNB-PtBA-(D)-4×Hex was treated with TFA according to the general procedure. ¹³C-NMR (100 MHZ, CD₃OD) δ 177.0, 172.2, 168.1, 163.9, 162.4, 159.4, 157.5, 153.0, 147.7, 135.7, 134.5, 132.0, 131.2, 129.2, 110.1, 109.1, 108.6, 108.0, 107.8, 106.2, 104.8, 95.9, 73.8, 69.6, 64.5, 61.3, 26.9, 26.5, 25.2, 24.1, 22.7, 22.1, 13.0, 11.4.

C7-PAA-(D)-4×Hex (C7-TBC): C7-PtBA-(D)-4×Hex was treated with TFA according to the general procedure. ¹³C-NMR (100 MHZ, CD₃OD) δ 180.0, 178.6, 172.6, 167.7, 154.5, 149.1, 131.3, 121.8, 107.8, 71.2, 65.9, 46.7, 46.0, 42.7, 36.3, 32.6, 29.8, 26.9, 26.7, 23.6, 14.5, 12.9.

### Example 2

### Self-Assembly of Tri-Block Amphiphiles

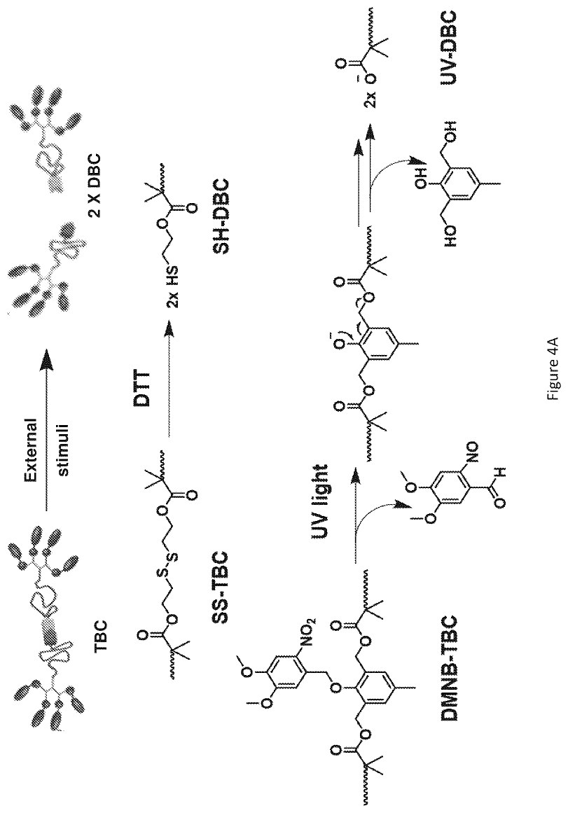

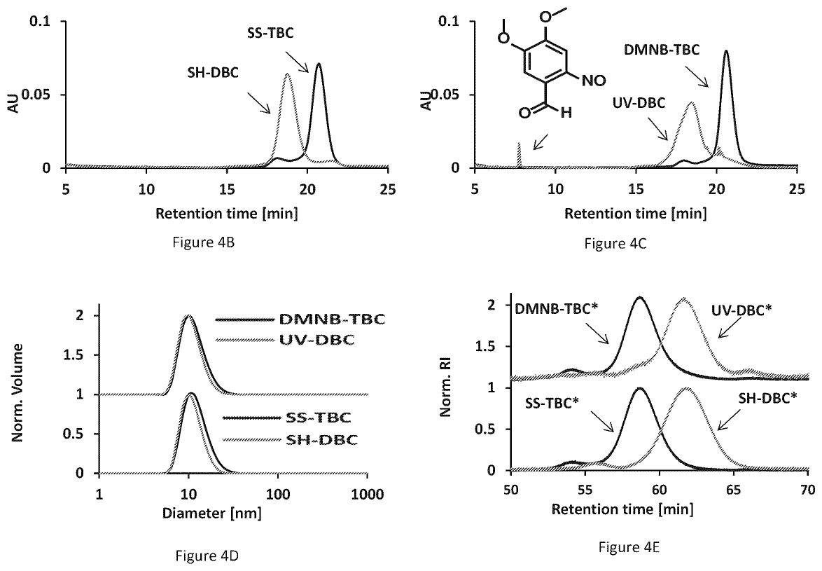

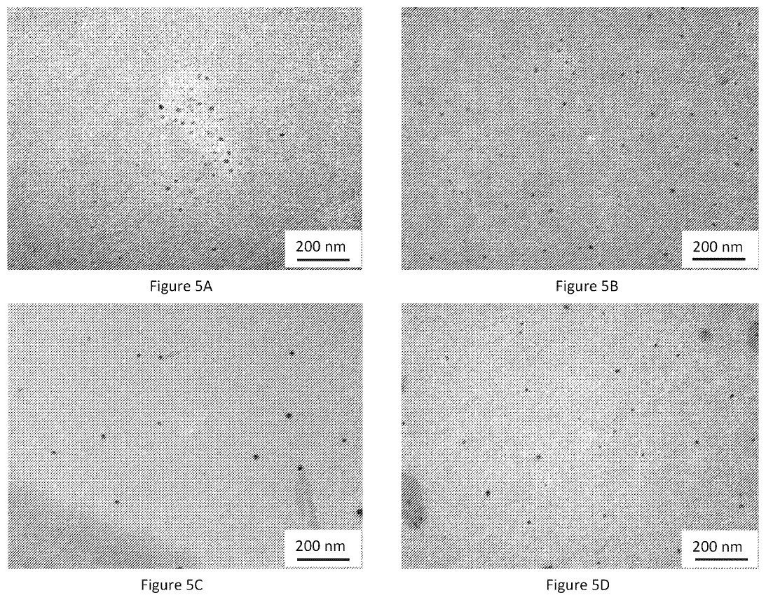

The self-assembly of the tri-block amphiphiles in aqueous media (PBS pH 7.4) was studied by dynamic light scattering (DLS). Structures with diameters of 12±4 nm for both SS- and DMNB-TBCs based micelles were observed (FIG. 4D). Further validation for the formation of micelles was obtained by transmittance electron microscopy (TEM), which showed spherical structures with similar diameters (FIGS. 5A-5B).

### Example 3

### TBS to DBC Transition

The architectural transition of self-assembled TBCs into DBCs was studied (FIG. 4A). First, HPLC analysis confirmed the splitting of the TBCs, showing the disappearance of the initial TBCs peaks and the appearance of new peaks with slightly shorter retention times, which were assigned as the DBCs (FIGS. 4B and 4C).

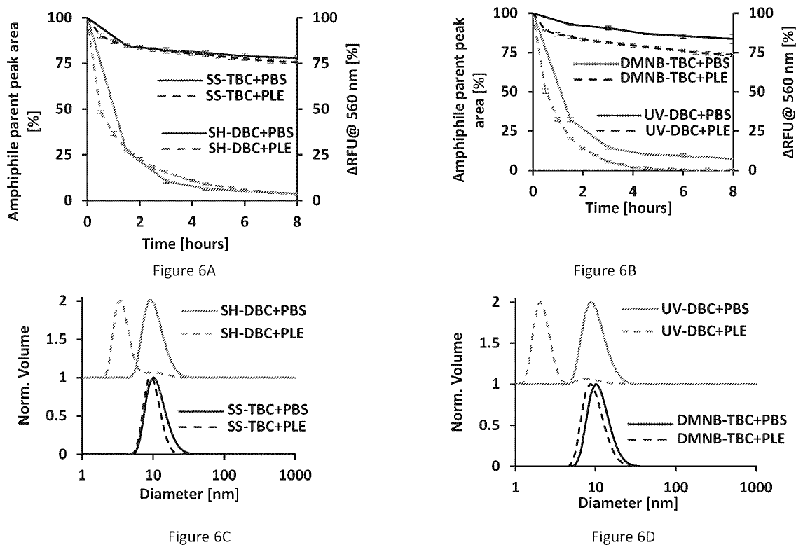

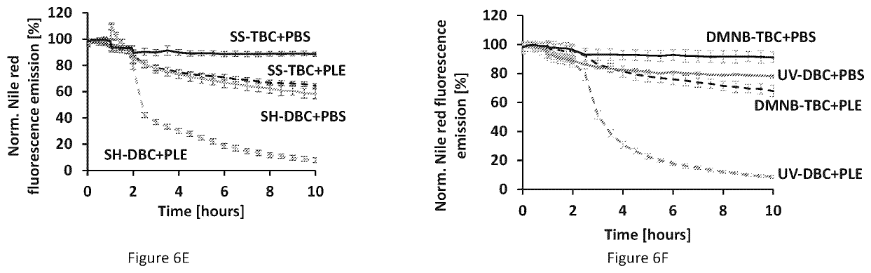

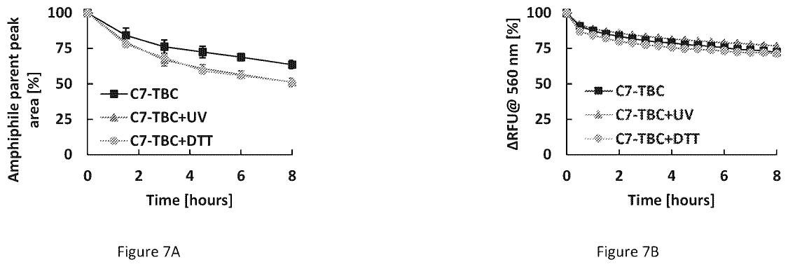

DLS was then used to determine micellar sizes of the DBCs directly after splitting (FIG. 4D) and after additional 8 hours of incubation at 37° C. (FIGS. 6C and 6D). The transformation into DBC amphiphiles did not lead to a significant change in micellar structures, as nearly identical diameters of 11±3 nm were observed for both SH- and UV-DBCs based micelles. This was also confirmed by TEM images (FIGS. 5C and 5D). The critical micelle concentrations (CMCs) were then determined using the Nile red method (Gillies et al., JACS 2004, 126 (38), 11936-11943) and were found to be around 3 μM for the two TBCs, while SH- and UV-DBC amphiphiles showed a slight increase to around 4-5 μM. This indicates that the architectural transition from TBC into DBC is not accompanied by a significant change in the thermodynamic stability of the micelles.

SEC was then used to verify the splitting of the amphiphiles. Due to strong column interaction of the multiple carboxylic acids of PAA, SEC was performed for the tert-butyl protected amphiphiles. SEC chromatograms confirmed the ability of the stimuli-responsive linkers to be cleaved, showing the disappearance of the TBCs and the appearance of DBCs with half the molecular weight (FIG. 4E).

### Example 4

### Enzymatic Degradation

The effect of TBCs to DBCs transition on the enzymatic degradation of the amphiphiles was then tested. Micellar solutions of both TBCs and DBCs were treated with PLE and incubated at 37° C. for 8 hours. The enzymatic degradation was quantified using HPLC by monitoring the peak areas of the amphiphiles at 420 nm, which is the absorbance wavelength of the labeling dye 7-DEAC. While SH-DBC amphiphiles were fully degraded within less than 4 hours (FIG. 6A, solid lines), SS-TBC showed only limited degree of degradation over 8 hours (˜25%). In parallel, the change in 7-DEAC fluorescence emission intensity at 560 nm, which is characteristic of the self-assembled state, was measured. Upon disassembly of the micelles, the diffusion of degraded hydrophilic polymers away from each other resulted in a decrease in excimer intensity (FIG. 6A, dashed lines). The fluorescence measurements were shown to correlate with the kinetic data obtained by HPLC, which indicates that indeed the enzymatic degradation led to micellar disassembly.

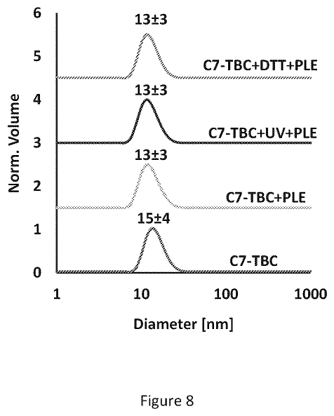

DLS measurements further confirmed the disassembly of the SH-DBC micelles, while the SS-TBC micelles remained intact in the presence of PLE (FIG. 6C). The photo-responsive DMNB-TBC and its corresponding DBC, showed very similar trends (FIGS. 6B and 6D). Importantly, when the control amphiphile C7-TBC, which lacks the cleavable linker was incubated with PLE alone or with PLE in the presence of DTT or after UV irradiation, only minor differences in enzymatic degradation rates and size were observed (FIGS. 7A-7B and 8). These control experiments demonstrate that the acceleration of the degradation rates was solely due to the splitting of the TBC amphiphiles and not because of background effects of the applied stimuli.

### Example 5

### Cargo Release by Enzymatic Degradation

The effect of the transition from TBC to DBC on the release of encapsulated hydrophobic cargo was studied. Nile red was selected as a model cargo as it is highly emissive in non-polar hydrophobic microenvironments such as a micelle, and has very weak emission in polar microenvironments such as PBS. Nile red was encapsulated within the TBC micelles to provide high fluorescence intensity. The TBC micelles were then incubated with DTT or UV irradiated in order to induce the splitting into DBCs, followed by addition of PLE. While only minor changes in fluorescence were observed for both types of TBC-based micelles in the presence of the activating enzyme, significant decreases in fluorescence emissions, indicating the release of Nile red, were observed for the DBC based micelles (FIGS. 6E and 6F). These results demonstrate that the splitting of the TBCs into DBCs can be used to control the enzymatic disassembly rates of the micelles and the induced cargo release.

### Example 6

TBC-DBC Interactions with a Transport Protein

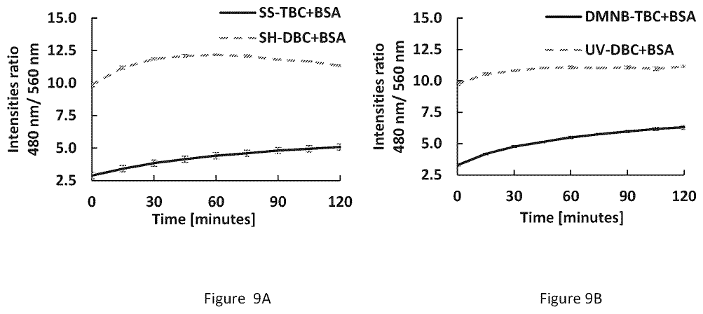

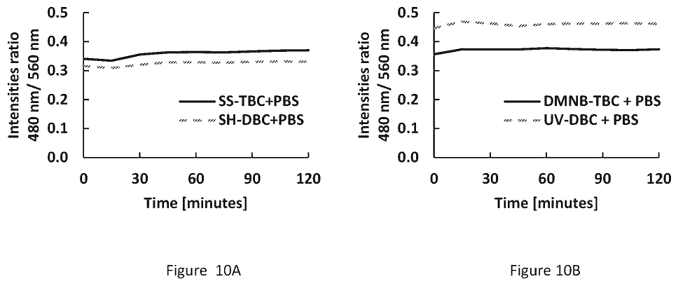

The interactions of TBCs and DBCs with bovine serum albumin (BSA) were studied. BSA is a transport protein that is known to interact with hydrophobic moieties. The self-reporting spectral mechanism of the micelles was utilized to evaluate differences in the degree of their interactions with BSA before and after splitting. Micellar solutions of TBCs and DBCs were treated with either BSA (FIGS. 9A-9B) or PBS (FIGS. 10A-10B) and fluorescence spectra were recorded every 15 minutes for 2 hours. To quantify the degree of micellar destabilization, the ratio between the fluorescence intensities at 480 nm and 560 nm, which correspond to 7-DEAC emissions in the amphiphiles' unimer and micellar states, respectively, were calculated. Interactions of BSA with the hydrophobic blocks of the amphiphiles shifts the unimer-micelle equilibrium towards the unimer state and in addition causes a significant increase in the fluorescence of the 7-DEAC dye due to a solvatochromic effect, causing an increase in the unimer/micelle emissions ratio. Based on the fluorescence spectra showing that the unimer/micelle fluorescence ratio for the TBC based micelles is roughly half the ratio of the DBC ones, it is clear that DBC micelles were more sensitive towards BSA interactions than TBC micelles. The comparable trends of the increased degree of interactions for both PLE (Example 5) and BSA with the DBC micelles, indicate that these protein-amphiphile interactions follow a similar mechanism. As the proteins cannot penetrate through the hydrophilic shell, the unimers are contemplated to escape the micelles in order to interact with them. Thus, the results indicate that the architectural change from TBD to DBC is useful for controlling the protein-responsiveness of the hydrophobic blocks.

Hence, fluorescently labeled TBC amphiphiles with enzymatically degradable dendritic end-groups and a single cleavable linker, which was located at the middle of the hydrophilic block were designed. An architectural change between TBC and DBC by a reducing agent or a UV light stimulus was shown not to affect the HLB of the splitted amphiphiles, the size of the micelles nor their thermodynamic properties. However, the stimuli-induced architectural transition from TBCs into two DBC amphiphiles, dramatically decreased the kinetic stability of the micelles towards interactions with enzymes. The ability to significantly affect the enzymatic responsiveness was demonstrated by the significantly faster enzymatic degradation and micellar disassembly of the DBC amphiphiles in comparison to the TBC micelles.

### Example 7

### TBC to DBC Enzyme-Induced Transition

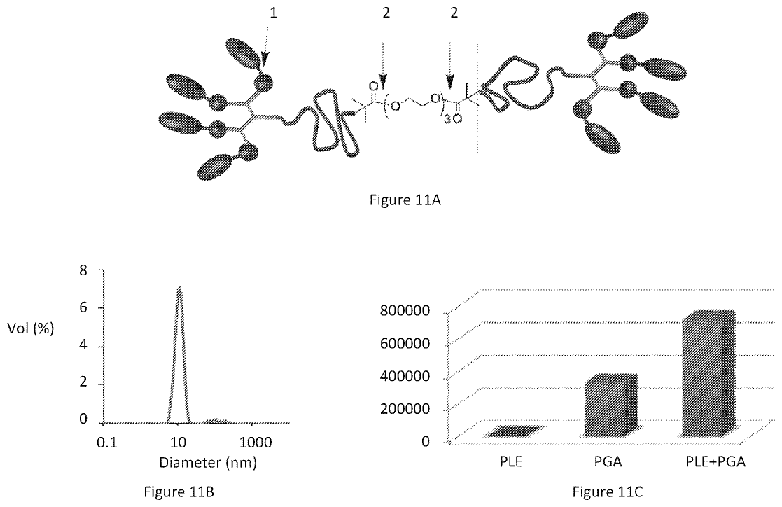

The feasibility of inducing tri-block to di-block transition by an activating enzyme was tested. Triethylene glycol was used for the preparation of a bi-functional ATRP initiator which was utilized for the synthesis of a tri-block copolymer amphiphile (FIG. 11A). DLS measurements showed the formation of structures with diameters of 10 nm (FIG. 11B). To test micelle degradation, the self-assembled amphiphiles were incubated in the presence of two enzymes, namely an esterase for inducing the cleavage of two ester bonds at the center of the hydrophilic polyacrylic acid chain (PLE), and an amidase for inducing the cleavage of amides located at the two hydrophobic end-groups (PGA). The cleavage induced by both enzymes was compared to the cleavage induced by a single enzyme (either the amidase or the esterase alone). A significantly faster degradation of the hydrophobic end-groups was shown to occur in the presence of both enzymes (PLE+PGA) as compared to the amidase enzyme alone (PGA). Furthermore, incubation with the esterase alone (PLA) did not lead to any cleavage of the hydrophobic end-groups (FIG. 11C). These results demonstrate the feasibility and applicability of the dual-activation mechanism according to the present invention towards the design of highly stable enzyme-responsive polymeric assemblies and their potential utilization as nano-carriers for drug delivery.

Taken together, the data show that the obstacle of maintaining the balance between stability and enzymatic-degradability of polymeric micelles has been overcome by a modular approach based on stimuli-induced splitting of amphiphilic tri-block copolymers into two substantially equivalent di-block amphiphiles. This architectural transition affects the kinetic stability of the assemblies as the faster unimer-micelle exchange rates of the smaller di-block amphiphiles makes their hydrophobic blocks more accessible to proteins. The amphiphiles of the present invention can therefore be used as nanocarriers that are activated by disease associated-enzymes for targeted delivery of drugs without compromising on the stability prior to degradation. The tri-block to di-block architectural transition provides extremely stable and yet highly responsive polymeric assemblies for various applications particularly in drug delivery where controlling the interaction between polymers and proteins is essential.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1-33. (canceled)

34. A delivery system in micellar form comprising micelles having an average particle size of about 5 nm to about 50 nm, the delivery system comprises a self-assembled amphiphilic tri-block copolymer comprising two substantially identical hydrophobic segments each comprising at least one protein cleavable site and a hydrophilic segment therebetween, wherein the hydrophilic segment comprises a central stimulus-responsive linker which undergoes cleavage upon a stimulus thereby forming two di-block copolymers which remain in micellar form, the two di-block copolymers having substantially the same hydrophilic to hydrophobic ratio as the amphiphilic tri-block copolymer.

35. The delivery system of claim 34, further comprising a cargo encapsulated within the micelles or a cargo covalently linked to the hydrophobic segments.

36. The delivery system of claim 35, wherein the cargo is selected from a pharmaceutical active ingredient, an agrochemical agent, a cosmetic agent, an imaging agent, and a diagnostic agent.

37. The delivery system of claim 34, wherein the amphiphilic tri-block copolymer is symmetric and the stimulus-responsive linker undergoes cleavage upon a stimulus at its center thereby forming two di-block copolymers which are identical.

38. The delivery system of claim 34, wherein the hydrophilic segment comprises polyacrylic acid, poly(2-hydroxyethyl acrylate), polyethylene glycol (PEG), or poly(oligo-ethylene glycol acrylate); or wherein the hydrophilic segment has a molecular weight of about 0.5 to about 100 kDa.

39. The delivery system of claim 34, wherein the hydrophilic segment is linked to the hydrophobic segments by a group selected from the group consisting of —Z—, —X¹—Z—X²—, —Z¹—X¹—Z²—X², wherein Z, Z¹, and Z² are each independently selected from C1-C10 alkylene, C2-C10 alkenylene, C2-C10 alkynylene, and arylene; X, X¹, and X² are each independently selected from —O—; —S—; —NH—; —C(═O)—; —C(═O)—O—; —O—C(═O)—O—; —C(═O)—NH—; —NH—C(═O)—NH—; —NH—C(═O)—O—; —S(═O)—; —S(═O)—O—; —PO(═O)—O—; triazolylene, and any combination thereof.

40. The delivery system of claim 34, wherein the linker in the hydrophilic segment is responsive to a chemically-induced stimulus, wherein the chemically-induced stimulus comprises the addition of a redox agent or an activating enzyme.

41. The delivery system of claim 40, wherein the redox agent comprises a reducing agent selected from the group consisting of dithiothreitol (DTT), thiol, glutathione, NADPH, and metal complexes; or wherein the redox agent comprises an oxidizing agent comprising a peroxide; or wherein the activating enzyme is selected from the group consisting of an amidase, an esterase, and an urease.

42. The delivery system of claim 34, wherein the linker in the hydrophilic segment is responsive to a physically-induced stimulus, wherein the physically-induced stimulus comprises a change in at least one of temperature, pH, light, and electric field.

43. The delivery system of claim 34, wherein the stimulus-responsive linker comprises a cleavable bond selected from a disulfide, a diselenide, an anhydride, an ester, an amide, an imine, an acetal, an urea, a thiourea, a hydrazone, an ether, a silyl ether, an oxyme, a boronic acid, a nitro, and an azo; or wherein the stimulus-responsive linker is covalently bound at its termini to the hydrophilic segment by a group selected from the group consisting of —Z—, —X¹—Z—X²—, —Z¹—X¹—Z²—X² wherein Z, Z¹, and Z² are each independently selected from C1-C10 alkylene, C2-C10 alkenylene, C2-C10 alkynylene, and arylene; X, X¹, and X² are each independently selected from O, S and NH; —O—; —S—; —NH—; —C(═O)—; —C(═O)—O—; —O—C(═O)—O—; —C(═O)—NH—; —NH—C(═O)—NH—; —NH—C(═O)—O—; —S(═O)—; —S(═O)—O—; —PO(═O)—O—; triazolylene, and any combination thereof.

44. The delivery system of claim 34, wherein the hydrophobic segments comprise hydrophobic dendrons.

45. The delivery system of claim 44, wherein the hydrophobic dendrons comprise between 0 to 5 generations; or wherein the hydrophobic dendrons comprise between 0 to 3 generations.

46. The delivery system of claim 45, wherein each generation of the hydrophobic dendron comprises a linear or branched C₁-C₂₀ alkylene, C₂-C₂₀ alkenylene, C₂-C₂₀ alkynylene or arylene moiety which is substituted at each end with a group selected from the group consisting of —O—, —S—, —NH—, —C(═O)—, —C(═O)—O—, —O—C(═O)—O—, —C(═O)—NH—, —NH—C(═O)—NH—, —NH—C(═O)—O—, —S(═O)—, —S(═O)—O—, —PO(═O)—O—, and any combination thereof; or wherein each generation of the hydrophobic dendron is derived from a compound having the following structure HX—Z—XH or HX—Z—CO₂H, wherein X is independently at each occurrence NH, S or O, and Z is selected from C1-C10 alkylene, C2-C10 alkenylene, C2-C10 alkynylene, and arylene; or wherein each generation of the hydrophobic dendron is derived from a compound selected from the group consisting of HX—CH₂—CH₂—XH, HX—(CH₂)₁₋₃—CO₂H, and HX—CH₂—CH(XH)—CH₂—XH wherein X is independently at each occurrence NH, S or O.

47. The delivery system of claim 34, wherein the protein cleavable site is an enzymatically cleavable site.

48. The delivery system of claim 47, wherein the enzymatically cleavable site comprises a functional group selected from the group consisting of an ester, an amide, a carbamate, a carbonate, a urea, a sulfate, an amidine, an ether, a phosphate, a phosphoamide, a sulfamate, a nitro, an azo, and a trithionate; or wherein the enzymatically cleavable site comprises a functional group represented by the structure of —O—C(O)—R′, —C(O)—OR′—NH—C(O)—R′ or —C(O)—NHR′ wherein R′ is C₁-C₁₂ alkyl or an aryl; or wherein the enzymatically cleavable site is cleavable by an amidase, an esterase, or a urease.

49. The delivery system of claim 34, wherein the protein is a transport protein; or wherein the protein is serum albumin.

50. The delivery system of claim 34, wherein the tri-block copolymer is represented by the structure depicted in FIG. 2B.

51. A method of preparing a tri-block copolymer amphiphile suitable as a delivery system according to claim 34, the method comprising the steps of:
(i) polymerizing a plurality of hydrophilic monomers at both termini of an activated stimulus-responsive linker to form a hydrophilic segment comprising a central stimulus-responsive linker;
(ii) functionalizing the termini of the hydrophilic segment; and
(iii) conjugating the functionalized hydrophilic segment with hydrophobic segments comprising at least one protein cleavable site.

52. A method of preparing a tri-block copolymer amphiphile suitable as a delivery system according to claim 34, the method comprising the steps of:
(i) reacting a stimulus-responsive linker activated at both termini with a hydrophilic polymer to form a hydrophilic segment comprising a central stimulus-responsive linker;
(ii) functionalizing the termini of the hydrophilic segment; and
(iii) conjugating the functionalized hydrophilic segment with hydrophobic segments comprising at least one protein cleavable site.

53. A method of delivering a cargo to a target site, the method comprising the steps of:
(i) providing a delivery system in micellar form comprising micelles having an average particle size of about 5 nm to about 50 nm, the delivery system comprises a cargo encapsulated within the micelles or covalently linked to the micelles and a self-assembled amphiphilic tri-block copolymer comprising two substantially identical hydrophobic segments each comprising at least one protein cleavable site and a hydrophilic segment therebetween, wherein the hydrophilic segment comprises a central stimulus-responsive linker;
(ii) applying a stimulus to induce cleavage of the stimulus-responsive linker thereby forming two di-block copolymers which remain in micellar form, the two di-block copolymers having substantially the same hydrophilic to hydrophobic ratio as the amphiphilic tri-block copolymer; and
(iii) contacting the di-block copolymers with a protein to induce cleavage of the protein cleavable site, thereby disassembling the micelles and releasing the cargo at the target site.
